(19) United States
(12) Patent Application Publication
(10) Pub. No.: US 2026/0240567 A1
(43) Pub. Date: Aug. 20, 2026

(54) PUNCTURING INSTRUMENT

(71) Applicant: FORCE ENGINEERING CO., LTD., Utsunomiya-shi, Tochigi (JP)

(72) Inventors: Takanori IMAI, Utsunomiya-shi (JP); Tomoaki HIGUCHI, Utsunomiya-shi (JP); Tomoyuki SAWAHATA, Mibu-machi (JP)

(73) Assignee: FORCE ENGINEERING CO., LTD., Utsunomiya-shi, Tochigi (JP)

(21) Appl. No.: 19/161,059

(22) PCT Filed: Feb. 7, 2024
(86) PCT No.: PCT/JP2024/004193

§ 371 Date: Aug. 29, 2025

(30) Foreign Application Priority Data
Mar. 10, 2023 (JP) 2023-037168

(51) Int. Cl. A61B 17/34

(52) U.S. Cl. CPC A61B 17/3498

(57) ABSTRACT

The present invention can prevent mispuncturing caused by the puncturing pressure applied to a needle body part during puncturing. A puncturing instrument 1 comprises a needle body part 2, a hub 3 that supports a base part of the needle body part 3, and a case 4 that accommodates the hub 3. Between the case 4 and the hub 3, the puncturing instrument 1 has an impact absorption body 4 that absorbs puncturing pressure during puncturing with the needle body part 3.

### TECHNICAL FIELD

The present invention relates to a puncturing instrument used for puncturing blood vessels and biological tissue.

### BACKGROUND ART

Conventionally, as a puncturing instrument used for puncturing blood vessels or biological tissue, a winged hypodermic needle 1 provided with an expansion/retraction control means (engagement claw portion 4k) has been known (for example, see Patent Literature 1). The expansion/retraction control means is provided to switch a state between a state for engaging and holding a wing portion 4 in a folded condition to restrict expansion of the wing portion 4 and retraction of a hub 3 and a needle main body 2 which is caused by the expansion of the wing portion 4 and a state for, in response to a predetermined engagement releasing manipulation, allowing the expansion of the wing portion 4 and the retraction of the hub 3 and the needle main body 2 which is caused by the expansion of the wing portion 4.

### CITATION LIST

### Patent Literature

Patent Literature 1: JP-B-6275303

### SUMMARY OF INVENTION

### Technical Problem

The winged hypodermic needle 1 according to Patent Literature 1 as described above includes the engagement claw portion 4k, which allows the state to be switched between the state for restricting the retraction of the needle main body 2 and the state for allowing the expansion of the wing portion 4 and the retraction of the hub 3 and the needle main body 4 which is caused by the expansion of the wing portion 4 in response to a predetermined engagement releasing manipulation. With this design, the wing portion is prevented from expanding before its use in contrary to the intension of an operator who performs hypodermic needle insertion, or the hub and the needle main body are prevented from retracting which is caused by the expansion of the wing portion, and thus the hypodermic needle can be prevented from being unusable. However, in this winged hypodermic needle 1, the wing portion 4 has to be expanded when inserting the needle main body 2, which may unnecessarily increase the puncture pressure applied to a puncture portion when inserting the needle main body 2. This is one of the problems to be solved by the present invention.

### Solution to Problem

The present invention has been made to solve the problems in view of the circumstances described above, and the invention according to claim 1 provides a puncturing instrument comprising: a needle main body; a hub that supports a base end portion of the needle main body; and a case body that houses the hub, wherein an impact absorption means for absorbing a puncture pressure during puncture of the needle main body is provided between the case body and the hub.

The invention according to claim 2 provides the puncturing instrument according to claim 1, wherein a locking mechanism that prevents the puncture pressure from being absorbed by the impact absorption means is releasably provided between the case body and the hub.

The invention according to claim 3 provides the puncturing instrument according to claim 2, wherein the hub is mounted rotatably relative to the case body, and the locking mechanism can be released by rotating the hub relative to the case body.

The invention according to claim 4 provides the puncturing instrument according to claim 2, wherein the locking mechanism is mounted removably relative to the case body, and the locking mechanism can be released by removing the locking mechanism from the case body.

The invention according to claim 5 provides the puncturing instrument according to claim 2, wherein the locking mechanism is mounted rotatably relative to the case body, and the locking mechanism can be released by rotating the locking mechanism relative to the case body.

The invention according to claim 6 provides the puncturing instrument according to claim 1, wherein the impact absorption means is formed of a member that is deformable during puncture of the needle main body.

The invention according to claim 7 provides the puncturing instrument according to claim 6, wherein the impact absorption means is formed of a member that does not restore when deformed.

The invention according to claim 8 provides the puncturing instrument according to claim 1, wherein the impact absorption means is formed with a piston mechanism, the piston mechanism includes: a cylinder portion provided in the case body; a piston portion, to which the needle main body is mounted, provided on a movable body that slidably fits relative to the case body and is assembled thereinto, so as to be movable within the cylinder portion; and a check valve provided in the piston portion, wherein the check valve restricts movement of air from a tip side space partitioned by the piston portion provided within the cylinder portion toward a base end side space, while allowing movement of the air from the base end side space toward the tip side space when the puncture pressure reaches a predetermined pressure.

The invention according to claim 9 provides the puncturing instrument according to claim 1, wherein the impact absorption means is formed with a ratchet mechanism, the ratchet mechanism includes a tooth portion provided on one of a sliding portion of the hub or that of the case body, and a claw potion provided on another one of the sliding portions to be locked with the tooth portion, the sliding portion of the hub and the sliding portion of the case body being designed to move relative to each other, and locking between the tooth portion and the claw portion are designed such that, for allowing relative movement of the case body, which has received the puncture pressure caused by the puncture of the needle main body, relative to the hub toward a tip side, the claw portion can forcibly pass beyond the tooth portion upon having received the puncture pressure, while, for restricting the relative movement thereof toward a base end side, the claw portion cannot pass beyond the tooth portion.

The invention according to claim 10 provides the puncturing instrument according to claim 9, wherein the tooth portion is formed on a hub side while the claw portion is formed on a case body side.

The invention according to claim 11 provides the puncturing instrument according to claim 10, wherein the tooth portion is designed to have multiple stages with a height increasing from a base end side to a tip side so that the puncture pressure generated when the claw portion forcibly passes beyond the tooth portion becomes higher at the tip side of the tooth portion.

### ADVANTAGEOUS EFFECTS OF INVENTION

According to claim 1, when the case body is gripped and then the needle main body is inserted into the portion to be inserted, the impact absorption means absorbs the puncture pressure applied to the needle main body. This enables prevention of erroneous puncture caused by the puncture pressure applied to the needle main body during puncture.

According to claim 2, providing the locking mechanism enables suppression of erroneous activation of the impact absorption means while the needle main body 2 is not used to puncture. Furthermore, inserting the needle main body into the portion to be inserted after the locking mechanism is released allows the impact absorption means to reliably absorb the puncture pressure applied to the needle main body.

According to claim 3, the locking mechanism is released by rotating the hub relative to the case body, which makes it possible to absorption of the puncture pressure by the impact absorption means. Thus, the locking mechanism can be released reliably and easily.

According to claim 4, the locking mechanism is released by removing the locking mechanism from the case body, which makes it possible to absorption of the puncture pressure by the impact absorption means. Thus, the locking mechanism can be released reliably and easily.

According to claim 5, the locking mechanism is released by rotating the locking mechanism relative to the case body, which makes it possible to absorption of the puncture pressure by the impact absorption means. Thus, the locking mechanism can be released reliably and easily.

According to claim 6, the impact absorption means is deformed by the puncture pressure applied to the needle main body during puncture of the needle main body, which causes the puncture pressure to be absorbed. This enables prevention of the reaction force on the needle main body when the impact absorption means absorbs the puncture pressure, and thus more appropriate absorption of the puncture pressure applied to the needle main body during puncture can be realized.

According to claim 7, the impact absorption means does not restore when deformed by the puncture pressure applied to the needle main body. This enables elimination of the reaction force on the needle main body when the impact absorption body absorbs the puncture pressure, and thus more appropriate absorption of the puncture pressure by the impact absorption body which is applied to the needle main body during puncture can be realized.

According to claim 8, the impact absorption means is formed with a piston mechanism including a cylinder portion provided in the case body, a piston portion, to which the needle main body is mounted, provided on a movable body that slidably fits relative to the case body and is assembled thereinto, so as to be movable within the cylinder portion, and a check valve provided in the piston portion, and the check valve restricts movement of air from a tip side space partitioned by the piston portion provided within the cylinder portion toward a base end side space, while allowing movement of the air from the base end side space toward the tip side space when the puncture pressure reaches a predetermined pressure. As a result, the check valve opens when the puncture pressure exceeds the pressure set in the check valve, which causes the piston portion to move and thus enables absorption of the puncture pressure. Therefore, puncture with appropriate puncture pressure can be realized.

According to claim 9, the impact absorption means is formed with a ratchet mechanism including a tooth portion provided on one of a sliding portion of the hub or that of the case body, and a claw potion provided on another one of the sliding portions to be locked with the tooth portion, the sliding portion of the hub and the sliding portion of the case body being designed to move relative to each other, and locking between the tooth portion and the claw portion are designed such that, for allowing relative movement of the case body, which has received the puncture pressure caused by the puncture of the needle main body, relative to the hub toward a tip side, the claw portion can forcibly pass beyond the tooth portion upon having received the puncture pressure, while, for restricting the relative movement thereof toward a base end side, the claw portion cannot pass beyond the tooth portion. As a result, in the case where the puncture pressure exceeds the load generated when the claw portion forcibly passes beyond the tooth portion, the claw portion forcibly passes beyond the tooth portion so that the puncture force can be absorbed. Therefore, puncture with appropriate puncture pressure can be realized.

According to claim 10, as the ratchet mechanism, the tooth portion is formed on a hub side while the claw portion is formed on a case body side. This enables simplification of manufacturing and structure.

According to claim 11, the tooth portion included in the ratchet structure is designed to have multiple stages with a height increasing from a base end side to a tip side, so that, in the process of puncture, during forcible passing at the base end side in the initial stage of puncture, absorption of a puncture resistance by a low puncture pressure is carried out, while, thereafter, absorption of a puncture resistance by a high puncture pressure can be carried out. Thus, adjustment of puncture pressure depending on the puncture condition can be realized.

### BRIEF DESCRIPTION OF DRAWINGS

FIG. 1 is a perspective view of a puncturing instrument according to the first embodiment.

FIG. 2 is an exploded perspective view of the puncturing instrument according to the first embodiment.

FIG. 3 is an axial cross-sectional view of the puncturing instrument according to the first embodiment in its locked state.

FIG. 4 is an axial cross-sectional view of the puncturing instrument according to the first embodiment in its free state.

FIG. 5 is an exploded perspective view of a puncturing instrument according to the second embodiment.

FIG. 6 is an axial cross-sectional view of the puncturing instrument according to the second embodiment in its locked state.

FIG. 7 is an axial cross-sectional view of the puncturing instrument according to the second embodiment in its free state.

FIG. 8 is a perspective view of a puncturing instrument according to the third embodiment in its locked state.

FIG. 9 is an exploded perspective view of the puncturing instrument according to the third embodiment.

FIG. 10 is a perspective view of the puncturing instrument according to the third embodiment in its free state.

FIG. 11 is a perspective view of a puncturing instrument according to the fourth embodiment.

FIG. 12 is an exploded perspective view of the puncturing instrument according to the fourth embodiment.

FIG. 13 (A) and (B) are a front view and a front sectional view of the puncturing instrument according to the fourth embodiment in its locked state, respectively.

FIG. 14 (A) and (B) are a top view and a top sectional view of the puncturing instrument according to the fourth embodiment in its locked state, respectively.

FIG. 15 (A) and (B) are a front view and a front sectional view of the puncturing instrument according to the fourth embodiment in its unlocked state, respectively.

FIG. 16 (A) and (B) are a front view and a front sectional view of the puncturing instrument according to the fourth embodiment in its intermediate movement state, respectively.

FIG. 17 (A) and (B) are a front view and a front sectional view of the puncturing instrument according to the fourth embodiment in its final movement state, respectively.

FIG. 18 (A) and (B) are a top view and a top sectional view of the puncturing instrument according to the fourth embodiment in its final movement state, respectively.

FIG. 19 is a perspective view of a puncturing instrument according to the fifth embodiment of the present invention.

FIG. 20 is an exploded perspective view of the puncturing instrument according to the fifth embodiment.

FIG. 21 (A) and (B) are a top view and a front sectional view of the puncturing instrument according to the fifth embodiment, respectively.

FIG. 22 (A), (B), and (C) are a front view, a top sectional view, and a side sectional view of the puncturing instrument according to the fifth embodiment in its locked state, respectively.

FIG. 23 (A) and (B) are a top view and a front sectional view of the puncturing instrument according to the fifth embodiment in its locked state, respectively.

FIG. 24 is a perspective view illustrating an unlocked state in which a holding member of the puncturing instrument according to the fifth embodiment is released from a locking body.

FIG. 25 is a perspective view illustrating a state in which a locking body of the puncturing instrument according to the fifth embodiment in a free state is locked with a holding member.

FIG. 26 (A) and (B) are a front view and a front sectional view of the puncturing instrument according to the fifth embodiment in the process of relative movement, respectively.

FIG. 27 (A) and (B) are a front view and a front sectional view of the puncturing instrument according to the fifth embodiment in the process of movement end, respectively.

FIG. 28 (A) and (B) are front sectional views illustrating a ratchet mechanism of the puncturing instrument according to the fifth embodiment in its locking state and forcible passing state, respectively.

FIG. 29 is a front sectional view of a main portion of a puncturing instrument according to the sixth embodiment.

FIG. 30 (A) and (B) are operation explanation diagrams, each illustrating an engagement state of the ratchet mechanism of the puncturing instrument according to the sixth embodiment.

### DESCRIPTION OF EMBODIMENTS

### First Embodiment

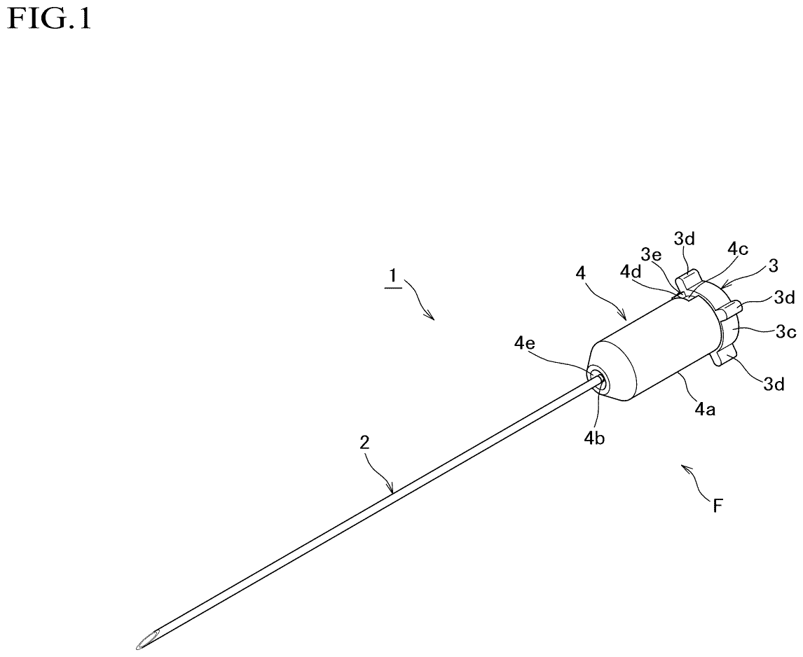

The first embodiment of the present invention will be described with reference to FIG. 1 to FIG. 4. A puncturing instrument 1 is a tool used for puncturing blood vessels or biological tissue, and includes a needle main body 2, a hub 3 supporting the base end of the needle main body 2, and a case body 4 in which the hub 3 is housed. The hub 3 has a substantially cylindrical hub main body 3a, and at the center of the tip side of the hub main body 3a, a needle insertion hole 3b, through which the base end of the needle main body 2 is inserted, is provided so as to pass through in the axial direction of the hub main body 3a. On the base end side of the hub main body 3a, a locking step portion 3c having the outer diameter dimension larger than that of the hub main body 3a is provided concentrically with the hub main body 3a. On the outer circumferential surface of the locking step portion 3c, multiple pieces, for example, four pieces of locking flange portions 3d are provided at equal intervals in the circumferential direction.

On the outer circumferential surface of the base end side of the hub main body 3a, a deformation prevention flange portion 3e, which protrudes in the radial direction of the hub main body 3a, is provided. Furthermore, the hub main body 3a includes multiple pieces, for example, four pieces of communicating holes 3f provided so as to pierce through the hub main body 3a along the axial direction thereof at the positions with the needle insertion hole 3b being centered. These communicating holes 3f are provided at the positions which are away from the needle insertion hole 3b by predetermined distances and equally separated from each other along the circumferential direction of the hub main body 3a. In the base end side of the needle insertion hole 3b of the hub 3, a mounting recess 3j having a circular cross-section is provided concentrically therewith. The mounting recess 3j is formed with the inner diameter larger than that of the needle insertion hole 3b, and is provided so as to communicate concentrically with the base end side of the needle insertion hole 3b. Furthermore, the mounting recess 3j is provided to extend from the base end side of the locking step portion 3c of the hub 3 to the middle position of the hub main body 3a, concentrically therewith. That is, the mounting recess 3j is used to push a plunger by receiving the tip of the cylinder end of a syringe (not illustrated) to inject the liquid in the syringe into the needle main body 2, or pull the plunger to collect blood and the like from the human body.

The case body 4 has a substantially cylindrical main body 4a with its tip side being tapered, and at the center of the tip side of the main body 4a, a needle insertion hole 4b, through which the needle main body 2 is inserted, is provided. The main body 4a has the inner diameter dimension substantially equal to the outer diameter dimension of the hub main body 3a of the hub 3, into which the hub main body 3a fits from the base end side thereof. Furthermore, on the opening edge of the base end side of the main body 4a, a locking recess 4c for locking the deformation prevention flange portion 3e of the hub 3 is provided. From this locking recess 4c, the base end edge of the main body 4a is cut out in a stepped manner in the circumferential direction of the main body 4a so as to form a sliding recess 4d. At the opening edge of the tip side of the needle insertion hole 4b of the case body 4, a tapered surface 4e is formed, and at the opening edge of the base end side of the needle insertion hole 4b, a tapered surface 4f is provided as well.

Between the case body 4 and the hub 3, a cylindrical impact absorption body 5 that serves as an impact absorption means for absorbing the puncture pressure generated during insertion of the needle main body 2, and a holding member 6 that fixes and holds the impact absorption body 5 at a predetermined position within the case body 4. The impact absorption body 5 has the outer diameter dimension equal to the inner diameter dimension of the main body 4a of the case body 4 and the outer diameter dimension of the hub main body 3a of the hub 3, and is made of an impact-absorbing material, such as a sponge material or an urethane material, which does not restore or rebound when compressed in the axial direction. Furthermore, the impact absorption body 5 is provided with a needle insertion hole 5a that passes through the needle insertion hole 3b of the hub main body 3a when the impact absorption body 5 is installed on the tip side of the hub main body 3a concentrically therewith, and communicating holes 5b that communicate with the communicating holes 3f of the hub main body 3a, respectively.

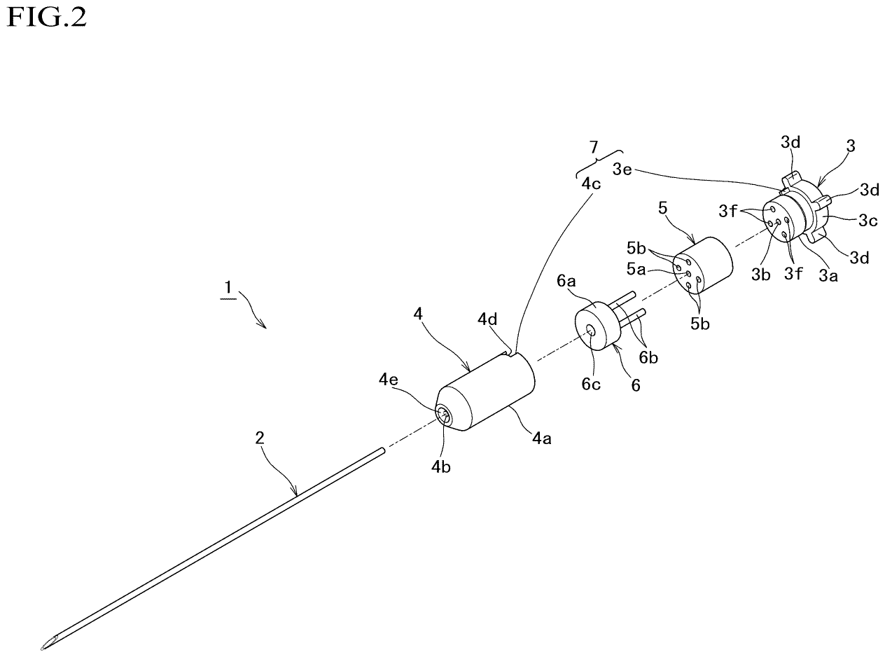

The holding member 6 includes a disk-shaped main body 6a and multiple pieces of, for example, four elongated cylindrical restricting flange portions 6b which protrude from the surface of the base end side of the main body 6a. The main body 6a has the outer diameter dimension equal to the inner diameter dimension of the main body 4a of the case body 4, and at the center of this main body 6a, a needle insertion hole 6c, through which the base end of the needle main body 2 is inserted and fixed, is provided along the axial direction thereof. Each of the restricting flange portions 6b protrudes from the base end surface of the main body 6a in the axial direction of the main body 6a, and has the length dimension equal to that in the axial direction of the impact absorption body 5 and has the outer diameter dimension equal to the inner diameter dimension of each of the communicating holes 5b of the impact absorption body 5. As illustrated in FIG. 2 to FIG. 4, the restricting flange portions 6b are mounted to such positions that they can pass through the communicating holes 5b of the impact absorption body 5, respectively, when the impact absorption body 5 is mounted to the base end side of the holding member 6.

Thus, the base end side of the needle main body 2 is inserted into the needle insertion hole 4b of the case body 4, the needle insertion hole 6c of the holding member 6, and the needle insertion hole 5a of the impact absorption body 5, in this order, so that it is fixed to the needle insertion hole 6c of the holding member 6 with being inserted into the needle insertion hole 3b of the hub 3. In this state, with the restricting flange portions 6b of the holding member 6 being inserted through the communicating holes 5b of the impact absorption body 5 from the tip side of the impact absorption body 5, the holding member 6 and the impact absorption body 5 are housed within the case body 4 of the main body 4a in this order such that the tip side of the main body 6a of the holding member 6 is positioned at the top, and the tip side of the hub main body 3a of the hub 3 fits into the base end side of the main body 5a and held in place to prevent it from being removed. At this time, the deformation prevention flange portion 3e of the hub 3 is locked by the locking recess 4c of the case body 4 so as to restrict the rotation of the hub 3 in the circumferential direction relative to the case body 4. Thus, the deformation prevention flange portion 3a and the locking recess 4c function as a locking mechanism 7. In the locking mechanism 7, the deformation prevention flange portion 3e of the hub 3 is locked by the locking recess 4c of the case body 4, which prevents the impact absorption body 5 from being deformed in the axial direction and thus locks (restricts) absorption of the puncture pressure generated during puncture of the needle main body 2.

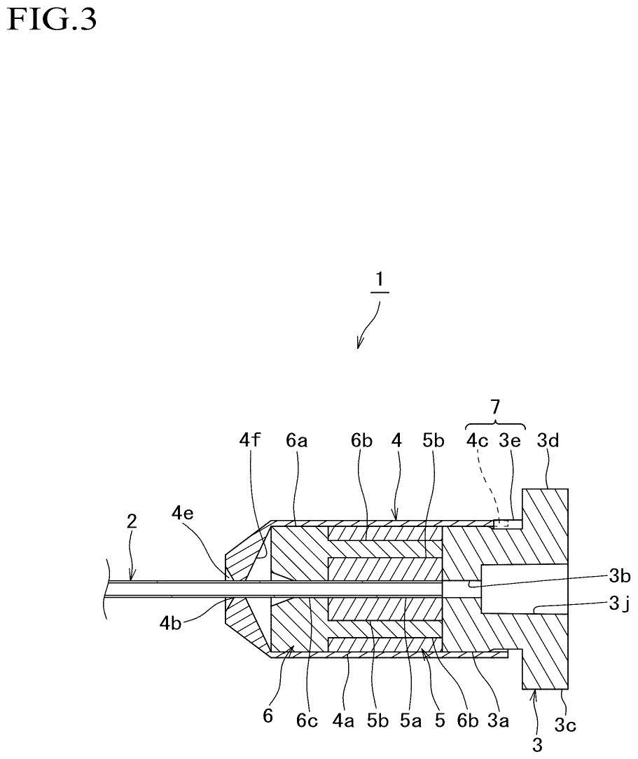

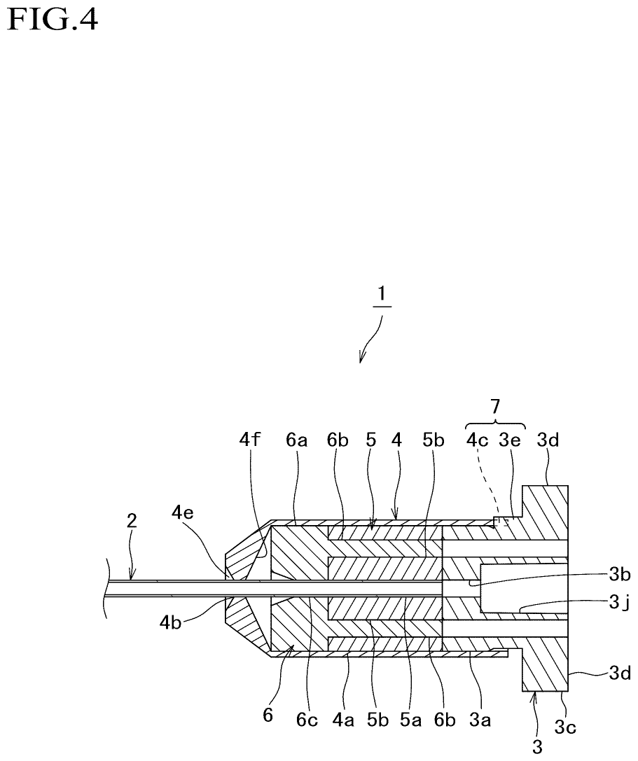

Furthermore, in the puncturing instrument 1, as illustrated in FIG. 3, in a locked state (stored state) R where the locking mechanism 7 is activated with the deformation prevention flange portion 3e of the hub 3 being locked by the locking recess 4c of the case body 4, the communicating holes 3f of the hub 3 are not communicated with the communicating holes 5b of the impact absorption body 5, respectively, so that the tips of the restricting flange portions 6b of the holding member 6 come into contact with the surface of the tip side of the hub main body 3a. This prevents the impact absorption body 5 from being deformed between the main body 6a of the holding member 6 and the hub main body 3a. On the other hand, in the puncturing instrument 1, as illustrated in FIG. 4, in a free state (using state) F where the locking mechanism 7 is released with the deformation prevention flange portion 3e of the hub 3 being rotated along the sliding recess 4d of the case body 4 in the circumferential direction thereof to release the locking of the deformation prevention flange portion 3e by the locking recess 4c of the case body 4, the communicating holes 3f of the hub 3 are communicated with the communicating holes 5b of the impact absorption body 5, respectively, so that the restricting flange portions 6b of the holding member 6 are communicated with the communicating holes 3f of the hub 3. This makes the impact absorption body 5 deformable between the main body 6a of the holding member 6 and the hub main body 3a in response to the puncture pressure (load) applied to the needle main body 2.

Next, a method of using the puncturing instrument 1 according to the first embodiment of the present invention will be described with reference to FIG. 1 to FIG. 4.

When inserting the puncturing instrument 1 into a target portion such as blood vessels or biological tissue of the human body, in the locked state R illustrated in FIG. 3, for example, the locking flange portion 3d of the hub 3 is gripped and rotated in the circumferential direction to make the hub 3 rotate relative to the case body 4, so that the deformation prevention flange portion 3e of the hub 3 moves from the locking recess 4c to the sliding recess 4d of the case body 4 and thus the free state F illustrated in FIG. 4 is established. In the free state F, the restricting flange portions 6b of the holding member 6 are communicated with the communicating holes 3f of the hub 3, which causes the impact absorption body 5 to be deformable between the main body 6a of the holding member 6 and the hub main body 3a of the hub 3.

In this state, for example, the locking flange portions 3d of the hub 3 of the puncturing instrument 1 are gripped, and then the needle tip of the needle main body 2 of the puncturing instrument 1 is inserted into a predetermined portion of the human body. At this time, for example, when the needle tip of the needle main body 2 punctures the surface of the target portion such as the skin tissue or biological tissue of the human body, a relatively large puncture pressure is applied to the needle main body 2 from the surface of the target portion, and this puncture pressure compresses (deforms) the target portion. At this time, in response to the puncture pressure, the restricting flange portions 6b of the holding member 6 are inserted into the communicating holes 3f of the hub 3, respectively, and the impact absorption body 5 is deformed between the main body 6a of the holding member 6 and the hub main body 3a of the hub 3. Thus, the puncture pressure applied to the needle main body 2 is absorbed by the deformation of the impact absorption body 5.

According to the first embodiment described above, in the free state F of the puncturing instrument 1 after being released from the locked state R, when the needle main body 2 is inserted into a target portion (portion to be inserted) of the human body, the puncture pressure applied to the needle main body 2 causes the impact absorption body 5 to be deformed, which enables the puncture pressure applied to the needle main body 2 to be absorbed. In other words, the pressure generated when the needle main body 2 of the puncturing instrument 1 passes through a target area, such as skin tissue, can be held to prevent the reaction force from being applied on the needle main body 2 when the impact absorption body 5 absorbs the puncture pressure. Therefore, erroneous puncture, such as posterior wall puncture, puncture of other tissues caused by tissue penetration, and the like can be prevented easily and appropriately.

Furthermore, the impact absorption body 5 is formed of a material that does not restore when deformed by the puncture pressure applied to the needle main body 2. This enables elimination of the reaction force on the needle main body 2, which is generated when the impact absorption body 5 absorbs the puncture pressure. Thus, more appropriate absorption of the puncture pressure by the impact absorption body 5 which is applied to the needle main body 2 during puncture can be realized.

In particular, using the puncturing instrument 1 when extracting eggs from the ovary of the human body in infertility treatment allows the puncture pressure generated when the needle main body 2 of the puncturing instrument 1 is inserted into the follicle to be absorbed. This can prevent unintended movement of the tip of the needle main body 2 caused by the puncture pressure, and thus damage to the follicle or excessive insertion of the needle main body 2. Thus, reduction of the burden on the human body and accurate extraction of eggs can be realized.

Still further, storing the puncturing instrument 1 in the locked state R enables elimination of unintended activation (deformation) of the impact absorption body 5 while the needle main body 2 is not used for puncture. Still further, switching the state of the puncturing instrument 1 from the locked state R to the free state F and then inserting the needle main body 2 into a target portion allows the impact absorption body 5 to reliably absorb the puncture pressure applied to the needle main body 2.

For switching the state of the puncturing instrument 1 from the locked state R to the free state F, the hub 3 is rotated relative to the case body 4 so that the deformation prevention flange portion 3e of the puncturing instrument 1 moves from the locking recess 4c to the sliding recess 4d of the case body 4. This causes the locking mechanism 7 to be released and the impact absorption body 5 to be ready to absorb the puncture pressure. Thus, the locking mechanism 7 can be released by this simple manipulation of rotating the hub 3 relative to the case body 4, which enables the locking mechanism 7 for keeping the locked state R of the puncturing instrument 1 to be reliably and easily released.

### Second Embodiment

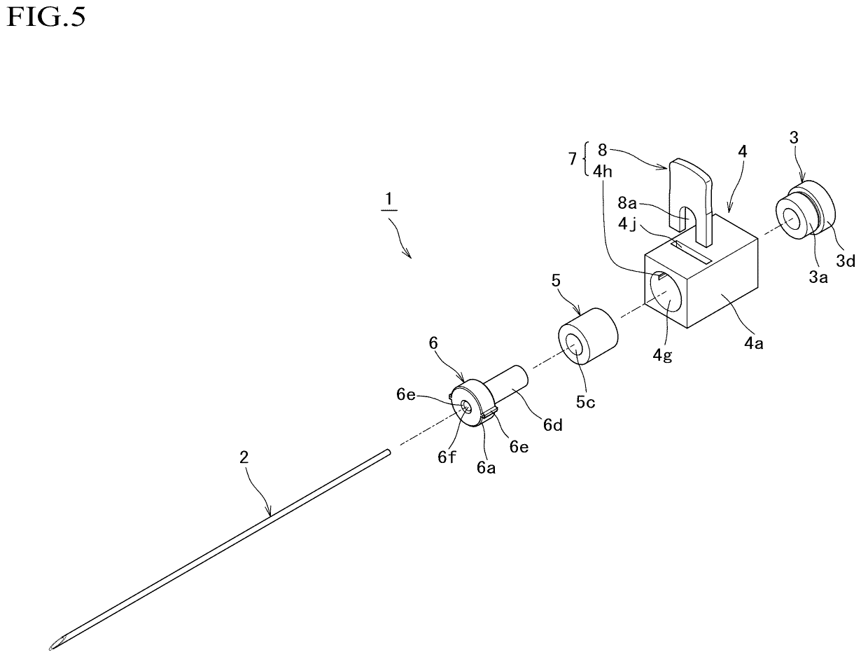

Next, the puncturing instrument 1 according to the second embodiment of the present invention will be described with reference to FIG. 5 to FIG. 7. The puncturing instrument 1 according to the second embodiment differs from the puncturing instrument 1 according to the first embodiment in its structure for bringing the impact absorption body 5 into the locked state R. In the following, the same functions are provided with the same reference signs as those in the first embodiment, and the differences from the first embodiment will be mainly described.

Specifically, in the puncturing instrument 1, the base end side of the needle main body 2 is held by the holding member 6. The holding member 6 includes the disk-shaped main body 6a and an elongated cylindrical sliding sleeve portion 6d mounted concentrically on the base end side of the main body 6a. The main body 6a has the outer diameter dimension larger than that of the sliding sleeve portion 6d, and on the circumferential surface of the main body 6a, a pair of ribs 6e is provided. The pair of ribs 6e is provided along the axial direction of the main body 6 a with being spaced apart from each other by 180° in the circumferential direction of the main body 6a.

At the center position of the main body 6a and sliding sleeve portion 6d of the holding member 6, a needle insertion hole 6f is formed to pass through along the axial direction of the main body 6a and sliding sleeve portion 6d. The base end side of the needle main body 2 is pressed into the needle insertion hole 6f from the main body 6a side of the needle insertion hole 6f and fixed thereto. The needle main body 2 is mounted such that the base end side of the needle main body 2 is housed inside more than the base end portion of the needle insertion hole 6f. Furthermore, on the base end side of the needle insertion hole 6f of the holding member 6, a mounting recess 6j having a circular cross-section is provided concentrically therewith. The mounting recess 6j has the inner diameter dimension larger than that of the needle insertion hole 6f, and is provided concentrically communicated with the base end side of the needle insertion hole 6f. Still further, the mounting recess 6j is provided concentrically so as to extend from the base end side of the sliding sleeve portion 6d to the middle position of the sliding sleeve portion 6d. In other words, the mounting recess 6j is also used to push a plunger by receiving the tip of the cylinder end of a syringe (not illustrated) to inject the liquid in the syringe into the needle main body 2, or pull the plunger to collect blood and the like from the human body.

The cylindrical impact absorption body 5 is mounted to the sliding sleeve portion 6d of the holding member 6. At the center position of the impact absorption body 5, a mounting hole 5c, to which the sliding sleeve portion 6d of the holding member 6 fits, is provided so as to pass through along the axial direction of the impact absorption body 5. In other words, the impact absorption body 5 has the outer diameter dimension equal to that of the main body 6a of the holding member 6 and the length shorter than that of the sliding sleeve portion 6d, and the inner diameter dimension of the mounting hole 5c is equal to the outer diameter dimension of the sliding sleeve portion 6d of the holding member 6.

The holding member 6 and the impact absorption body 5 are slidably held within the case body 4. The case body 4 includes the prismatic main body 4a having the length dimension larger than that of the holding member 6 along the axial direction. At the center of the main body 4a, an insertion hole 4g is provided so as to pass through along the longitudinal direction. The insertion hole 4g has the inner diameter dimension equal to the outer diameter dimension of the main body 6a of the holding member 6.

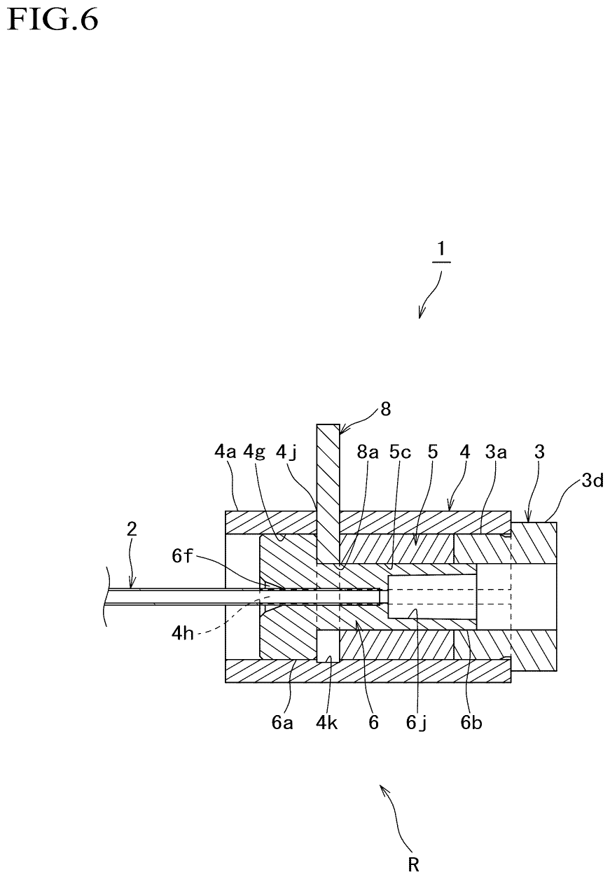

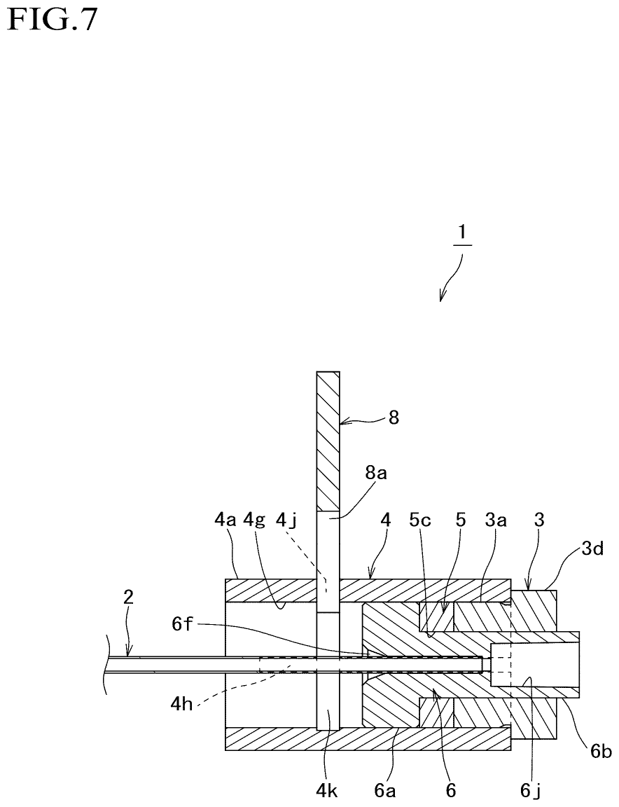

On a circumferential surface portion of the insertion hole 4g, a pair of slit grooves 4h is formed along the longitudinal direction of the insertion hole 4g. Each of the pair of slit grooves 4h is formed into a straight line so as to extend from the position shifted by a predetermined distance from the tip side of the main body 4a toward the base end side of the main body 4a. The slit grooves 4h are provided on both the sides in the width direction of the main body 4a, respectively, and are spaced apart from each other by 180° in the circumferential direction of the main body 4a. Furthermore, on the upper surface of the main body 4a of the case body 4, a mounting groove 4j is provided so as to pass therethrough. The mounting groove 4j is provided so as to extend along the width direction of the main body 4a, pass through from the upper surface of the main body 4a toward the insertion hole 4g, and extend along the radial direction of the insertion hole 4g. On the lower surface side of the insertion hole 4g facing the mounting groove 4j of the case body 4, a fitting reception portion 4k is provided. The fitting reception portion 4k is provided along the width direction of the main body 4a, in other words, along the radial direction of the insertion hole 4g, as illustrated in FIG. 6 and FIG. 7.

In the case body 4, a flat-plate-shaped locking plate 8 is removably inserted into the mounting groove 4j and mounted thereto. The locking plate 8 has the thickness dimension equal to the width dimension of the mounting groove 4j, and has the width dimension equal to the length dimension of the mounting groove 4j. Furthermore, in the locking plate 8, a fitting recess 8a is formed by cutting the lower end side of the locking plate 8 into a concave shape. The fitting recess 8a has the width dimension equal to the outer diameter dimension of the sliding sleeve portion 6d of the holding member 6. In other words, in the locking plate 8, the lower side of locking plate 8 fits and is inserted into the mounting groove 4j of the case body 4, and in this state, the fitting recess 8a fits to the sliding sleeve portion 6d of the holding member 6 which has been inserted through the insertion hole 4g of the case body 4 and mounted thereto, and also a lower end portion of the locking plate 8 fits to the fitting reception portion 4k of the case body 4. The locking plate 8 thus designed restricts movement of the holding member 6 toward the base end side to prevent deformation of the impact absorption body 5.

The hub 3 is mounted to the base end side of the case body 4. The hub 3 includes the hub main body 3a having the outer diameter dimension equal to the inner diameter dimension of the insertion hole 4g of the case body 4. On the base end side of the hub main body 3a, a locking step portion 3d having the outer diameter dimension larger than the inner diameter dimension of the insertion hole 4g of the case body 4 is provided concentrically therewith. The hub 3 is mounted such that the hub main body 3a fits to the insertion hole 4g of the case body 4 from the base end side of the case body 4 and the locking step portion 3c is locked with the peripheral edge of the base end side of the insertion hole 4g.

Thus, the needle main body 2 is fixed to the holding member 6 with the base end side of the needle main body 2 is pressed into the needle insertion hole 6f of the holding member 6. In this state, in the case body 4, the ribs 6e of the holding member 6 slidably fit into the slit grooves 4h of the case body 4, respectively, and the holding member 6 fits and housed from the base end side of the insertion hole 4g of the case body 4. In the state where the holding member 6 fits into the tip of the case body 4, the locking plate 8 is inserted into the mounting groove 4j of the case body 4. Thus, the fitting recess 8a of the locking plate 8 fits to the tip-side position of the sliding sleeve portion 6d of the holding member 6, and also the lower end portion of the locking plate 8 fits to the fitting reception portion 4k of the case body 4.

As a result, the holding member 6 is held between the locking plate 8 and the edges of the tips of the slit grooves 4h of the case body 4, which functions as the locking mechanism 7 for restricting movement of the holding member 6 toward the base end side. In the locking mechanism 7, the fitting recess 8a of the locking plate 8 fits to the sliding sleeve portion 6d of the holding member 6, so as to restrict movement of the holding member 6 toward the base end side, in other words, deformation of the impact absorption body 5, and lock the absorption of the puncture pressure generated during insertion of the needle main body 2. In this state, the impact absorption body 5 fits such that the tip side of the impact absorption body 5 comes into contact with the locking plate 8 from the base end side of the insertion hole 4g of the case body 4. In the impact absorption body 5, the sliding sleeve portion 6d of the holding member 6 fits to the mounting hole 5c of the impact absorption body 5. Furthermore, the hub 3 fits from the base end side of the case body 4, the hub main body 3a of the hub 3 fits into the insertion hole 4g of the case body 4, and the locking flange portions 3d of the hub 3 is locked to the peripheral edge of the base end side of the insertion hole 4g of the case body 4.

In the puncturing instrument 1, as illustrated in FIG. 6, in the locked state R in which the fitting recess 8a of the locking plate 8 fits to the tip side of the sliding sleeve portion 6d of the holding member 6 and the lower end portion of the locking plate 8 fits to the fitting reception portion 4k of the case body 4 to activate the locking mechanism 7, movement of the holding member 6 toward the base end side is restricted between the locking plate 8 and the tips of the edges of the slit grooves 4h of the case body 4, so as to prevent deformation of the impact absorption body 5 caused by the puncture pressure from the tip side via the needle main body 2. On the other hand, in the puncturing instrument 1, as illustrated in FIG. 7, in the free state F in which the locking mechanism 7 is released by, for example, moving the locking plate 8 upward to remove it from the case body 4, the holding member 6 becomes movable toward the base end side along the slit grooves 4h of the case body 4, which causes the impact absorption body 5 to be deformable between the main body 6a of the holding member 6 and the hub main body 3a of the hub 3 in response to the puncture pressure applied to the needle main body 2.

In this state, for example, the case body 4 of the puncturing instrument 1 is gripped, and then the needle tip of the needle main body 2 of the puncturing instrument 1 is inserted into a predetermined portion of the human body. The puncture pressure generated when the needle tip of the needle main body 2 is inserted into the predetermined portion of the human body causes the impact absorption body 5 to be deformed between the main body 6a of the holding member 6 and the hub main body 3a of the hub 3, and the puncture pressure applied to the needle main body 2 is absorbed by this deformation of the impact absorption body 5.

According to the second embodiment described above, when the needle tip of needle main body 2 is inserted into a target portion of the human body in the state where the puncturing instrument 1 is brought into the free state F from the locked state, the impact absorption body 5 is deformed by the puncture pressure applied to the needle main body 2. Therefore, the same advantageous effects as those of the puncturing instrument 1 according to the first embodiment can be obtained. Furthermore, the state of the puncturing instrument 1 can be switched from the locked state R to the free state F by pulling the locking plate 8 upward from the case body 4 to remove it, which enables the locking mechanism 7 for keeping the locked state R of the puncturing instrument 1 to be reliably and easily released.

### Third Embodiment

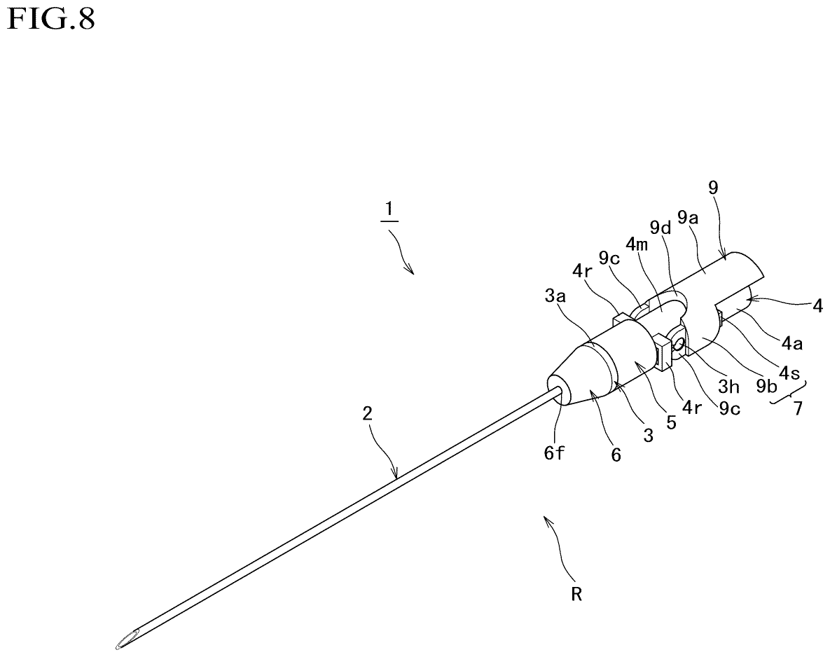

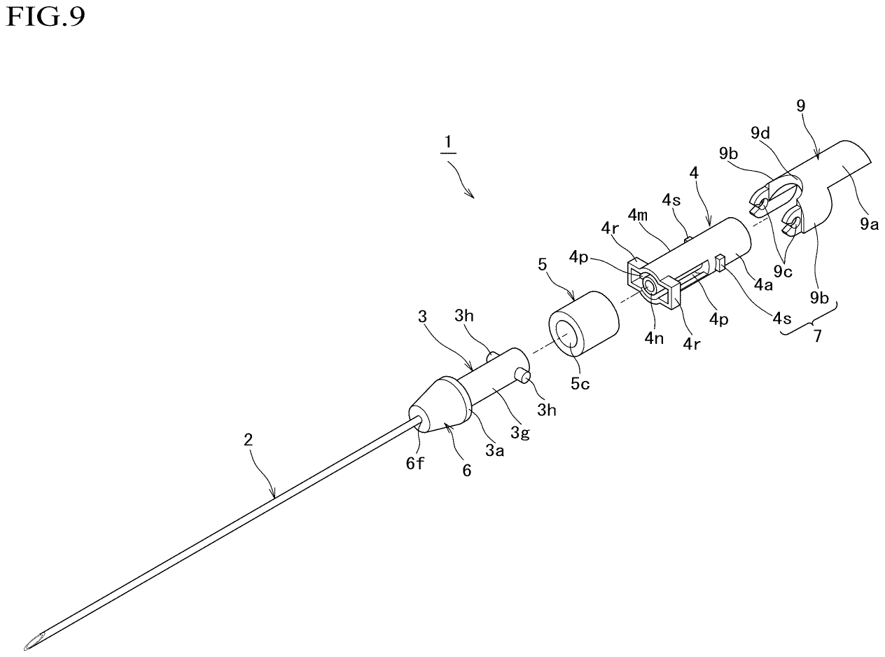

Next, the puncturing instrument 1 according to the third embodiment of the present invention will be described with reference to FIG. 8 to FIG. 10. The puncturing instrument 1 according to the third embodiment differs from the puncturing instrument 1 according to the first embodiment in its structure for bringing the impact absorption body 5 into the locked state R. In the following, the same functions are provided with the same reference signs as those in the first embodiment, and the differences will be mainly described.

Specifically, in the puncturing instrument 1 according to the third embodiment, the base end side of the needle main body 2 is held by the holding member 6. The holding member 6 is formed in a conical shape that tapers toward the tip side. The hub 3 is mounted to the base end side of the holding member 6. The hub 3 includes the cylindrical hub main body 3a having the outer diameter dimension equal to that of the base end side of the holding member 6, and the sliding sleeve portion 3g mounted concentrically on the base end side of the hub main body 3a. At the center of the holding member 6 and that of the hub 3, the needle insertion holes 6f, 3b are provided along the axial direction.

The needle main body 2 is mounted such that the base end thereof protrudes from a base end portion of the hub 3 toward the base end side. On a position near the base end of the outer circumferential surface of the sliding sleeve portion 3g of the hub 3, a pair of rotatable support portions 3h is mounted. The pair of rotatable support portions 3h is formed as cylindrical projections extending along the radial direction of the sliding sleeve portion 3g, at positions spaced apart from each other by an equal interval, in other words, by 180°, in the circumferential direction of the sliding sleeve portion 3g. At the base end side of the sliding sleeve portion 3g, a mounting recess (not illustrated) having a circular cross-section is provided concentrically therewith as well. This mounting recess is also used to push a plunger by receiving the tip of the cylinder end of a syringe (not illustrated) to inject the liquid in the syringe into the needle main body 2, or pull the plunger to collect blood and the like from the human body. The cylindrical impact absorption body 5 is mounted to the sliding sleeve portion 3g. The impact absorption body 5 has the length dimension smaller than the distance from the tip side of the sliding sleeve portion 3g of the hub 3 to the rotatable support portions 3h. At the center of the impact absorption body 5, the mounting hole 5c having the inner diameter dimension equal to the outer diameter dimension of the sliding sleeve portion 3g is provided so as to pass through along the axial direction of the impact absorption body 5. In other words, the impact absorption body 5 is mounted so as to fit into the sliding sleeve portion 3g which is positioned closer to the tip side than the rotatable support portions 3h of the hub 3.

The sliding sleeve portion 3g of the hub 3 is mounted so as to fit from the tip side of the case body 4. The impact absorption body 5 is mounted so as to be sandwiched between the main body 3a of the hub 3 and the case body 4. This case body 4 includes the cylindrical main body 4a and a cylindrical fitting cylinder potion 4m mounted concentrically at the front end side of the main body 4a. The fitting cylinder potion 4m has the outer diameter dimension equal to that of the main body 4a, and has the outer diameter dimension equal to that of the impact absorption body 5. Inside of the fitting cylinder potion 4m, a cylindrical sliding reception portion 4n, into which the sliding sleeve portion 3g of the hub 3 fits, is formed concentrically therewith. The sliding reception portion 4n has the length dimension equal to that of the fitting sleeve portion 4m and has the outer diameter dimension equal to the inner diameter dimension of the sliding sleeve portion 3g of the hub 3.

The fitting sleeve portion 4m of the case body 4 includes a pair of sliding slit grooves 4p, which is formed by cutting the opening edge at the tip side of the fitting sleeve portion 4m into a concave shape toward the base end side thereof. The pair of sliding slit grooves 4p is provided at positions spaced apart from each other by an equal interval, in other words, by 180°, along the circumferential direction of the fitting sleeve portion 4m. Furthermore, on the opening edge of the tip side of the fitting sleeve portion 4m, a pair of U-shaped locking recesses 4r is provided so as to cover the tip portions of the sliding slit grooves 4p. Each of the pair of locking recesses 4r is shaped so as to receive the rotational support portions 3h of the hub 3 passing therethrough when the hub 3 fits thereinto from the tip side of the case body 4. Furthermore, a pair of locking flange portions 4s is provided so as to protrude from the circumferential surface of the main body 4a of the case body 4 at the position closer to the tip thereof. The pair of locking flange portions 4s is provided in a row along the axial direction with a predetermined distance therebetween near the base end side of the sliding slit grooves 4p.

A manipulation member 9 for bringing the puncturing instrument 1 into the locked state is mounted to the fitting sleeve portion 4m of the case body 4.

The manipulation member 9 includes an arc-shaped manipulation portion 9a having the inner diameter dimension equal to the outer diameter dimension of the main body 4a of the case body 4. On both the sides of the tip of the manipulation portion 9a, a pair of holding flange portions 9b is provided. In each of the pair of holding flange portions 9b, the side surface of the base end side is formed in an arc shape. Furthermore, on the tip side of the holding flange portions 9b, substantially C-shaped rotational reception portions 9c, which are rotatably fit into the rotatable support portions 3h of the hub 3, are provided. The pair of rotational reception portions 9c is positioned on both the sides of the manipulation member 9, respectively, and mounted at the positions spaced apart from each other by an equal distance, in other words, by 180°, along the circumferential direction of the manipulation member 9. Furthermore, at the center portion of the tip side of the manipulation member 9, a fitting recess 9d having the inner diameter dimension equal to the outer diameter dimension of the main body 4a of the case body 4 is provided. The fitting recess 9d is shaped so as to fit thereto from the upper side of the fitting sleeve portion 4m of the case body 4 when the manipulation member 9 is rotated toward the tip side around the pair of rotational reception portions 9c so as to be stood.

Thus, the needle main body 2 is mounted such that the base end side thereof is inserted through the needle insertion hole 6c of the holding member 6, and subsequently the needle insertion hole 3b of the hub 3. The impact absorption body 5 is mounted to the sliding sleeve portion 3g between the main body 3a and the rotating support portions 3h of the hub 3. In this state, the sliding sleeve portion 3g of the hub 3 fits from the tip side of the case body 4 between the fitting sleeve portion 4m and the sliding reception portion 4n, and is mounted thereto. In this state, the rotatable support portions 3h of the hub 3 are mounted with fitting into the locking recesses 4r of the case body 4 and being moved toward the base end side along the sliding slit grooves 4p of the case body 4. In the case body 4, the fitting recess 9d for the manipulation member 9 fits into the fitting sleeve 4m of the case body 4 from the upper side of the case body 4, and each of the rotational reception portions 9c of the manipulation member 9 fits to the rotational support portions 3h of the hub 3. In this state, the manipulation member 9 is rotated toward the base end side with each of the rotational reception portions 9c being as the rotation center, and mounted such that the lower surface side of the manipulation portion 9a is contacted with the upper side of the main body 4a of the case body 4.

As a result, as illustrated in FIG. 8, the impact absorption body 5 is held between the main body 3a of the hub 3 and the pair of locking recesses 4r of the case body 4, and this holding state is maintained by the contact between the pair of locking recesses 4r of the case body 4 and the rotational reception portions 9c of the manipulation member 9, as well as the contact between the pair of holding flange portion 9b of the manipulation member 9 and the locking flange portions 4s of the case body 4, which functions as the locking mechanism 7 that restricts movement of the hub 3 toward the base end side.

In the puncturing instrument 1, in the locked state R where the manipulation portion 9a of the manipulation member 9 is brought into contact with the main body 4a of the case body 4 to activate the locking mechanism 7, the rotational reception portions 9c of the manipulation member 9 come in contact with the pair of locking recesses 4r of the case body 4, and also the pair of holding flange portions 9b of the manipulation member 9 comes in contact with the locking flange portions 4s of the case body 4. In the puncturing instrument 1 thus designed above, movement of the hub 3 toward the base end side is restricted, which enables prevention of deformation of the impact absorption body 5 caused by the puncture pressure via the needle main body 2 and the hub 3.

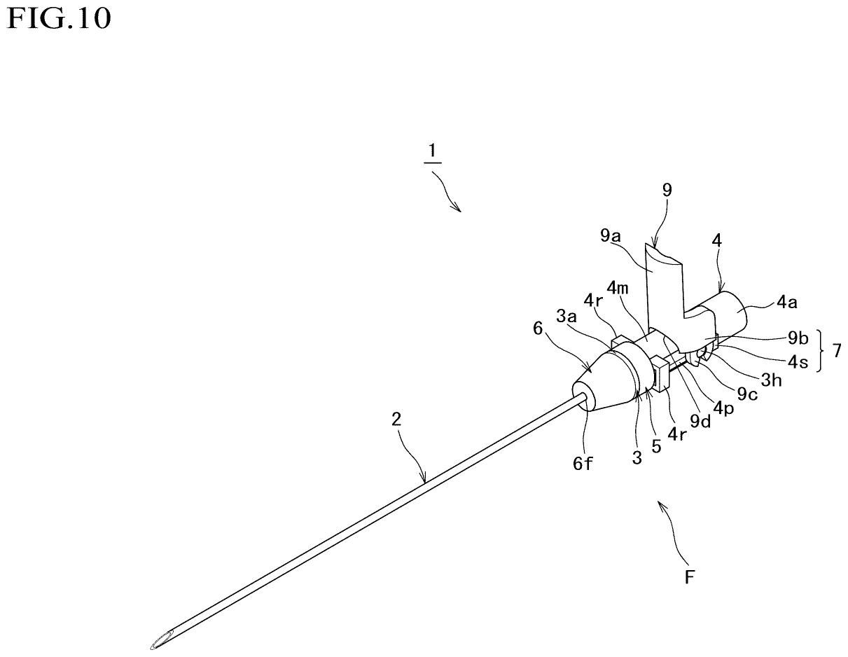

As illustrated in FIG. 10, in the puncturing instrument 1, for example, the manipulation portion 9a of the manipulation member 9 is gripped and the manipulation member 9 is rotated upward until the fitting recess 9d of the manipulation member 9 fits into the fitting sleeve portion 4m of the case body 4, so as to release the locking mechanism 7 and bring it into the free state F. As a result, in the puncturing instrument 1, restriction on movement of the hub 3 toward the base end side is released by the contact between the pair of holding flange portions 9b of the manipulation member 9 and the locking flange portions 4s of the case body 4, which allows the hub 3 to move toward the base end side along the sliding slit grooves 4p of the case body 4, and thus causes the impact absorption body 5 to be deformable between the hub main body 3a of the hub 3 and the locking recesses 4r of the case body 4 in response to the puncture pressure applied to the needle main body 2.

In this state, when the needle tip of the needle main body 2 of the puncturing instrument 1 is inserted into a predetermined portion of the human body, the puncture pressure applied to the needle tip of the needle main body 2 causes the impact absorption body 5 to be deformed between the hub 3 and the case body 4, and the puncture pressure applied to the needle main body 2 is absorbed by this deformation of the impact absorption body 5.

According to the third embodiment described above, when the needle tip of needle main body 2 is inserted into a target portion of the human body while bringing the puncturing instrument 1 into the free state F from the locked state, the impact absorption body 5 is deformed by the puncture pressure applied to the needle main body 2. Therefore, the same advantageous effects as those of the puncturing instrument 1 according to the first embodiment can be obtained. Furthermore, the puncturing instrument 1 can be switched from the locked state R to the free state F by rotating the manipulation member 9 to make it stand. This enables a manipulation for releasing the locking mechanism 7 that keeps the puncturing instrument 1 in the locked state R to be visually recognized, and thus the manipulation for releasing the locking mechanism 7 to be performed reliably and easily.

### Fourth Embodiment

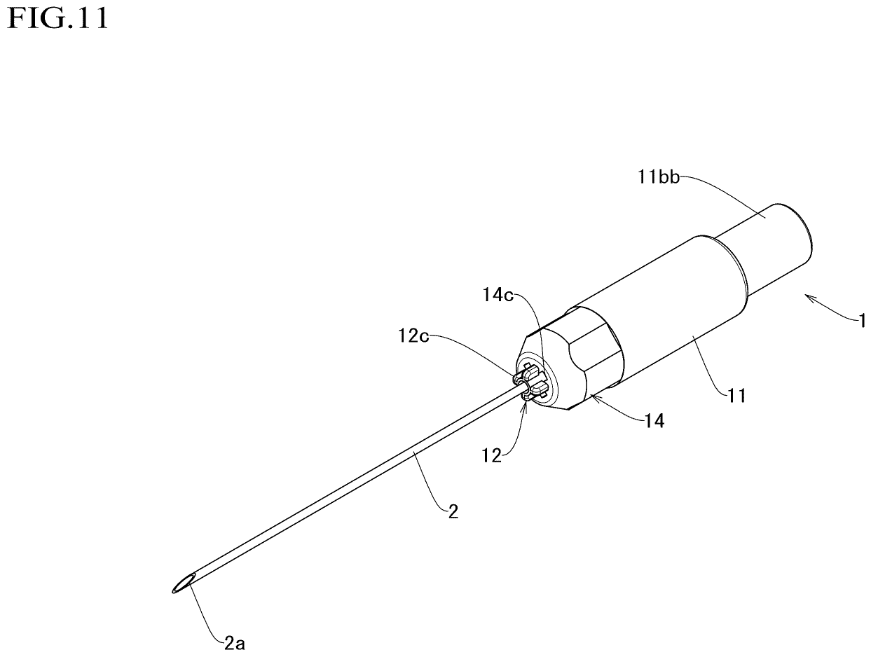

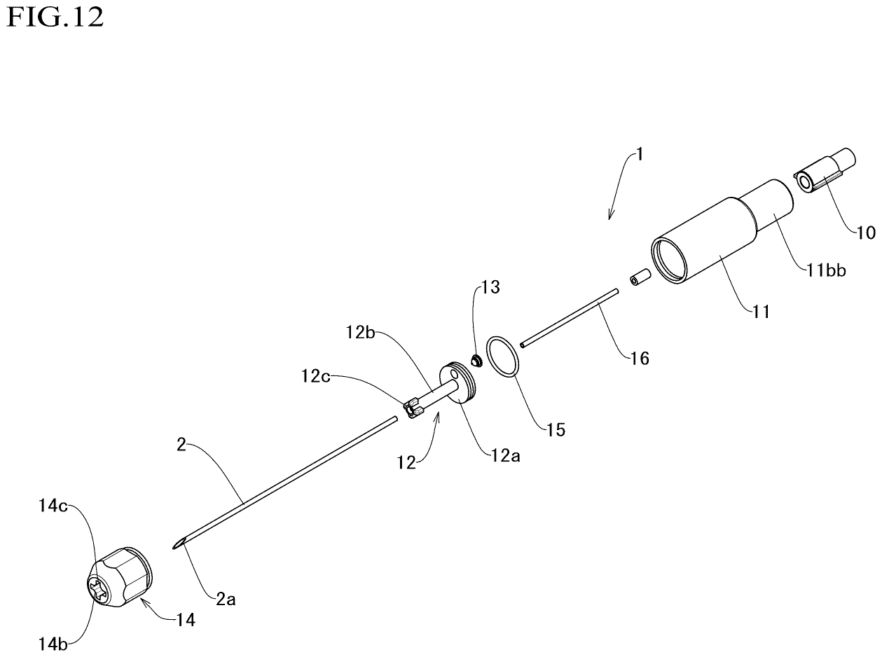

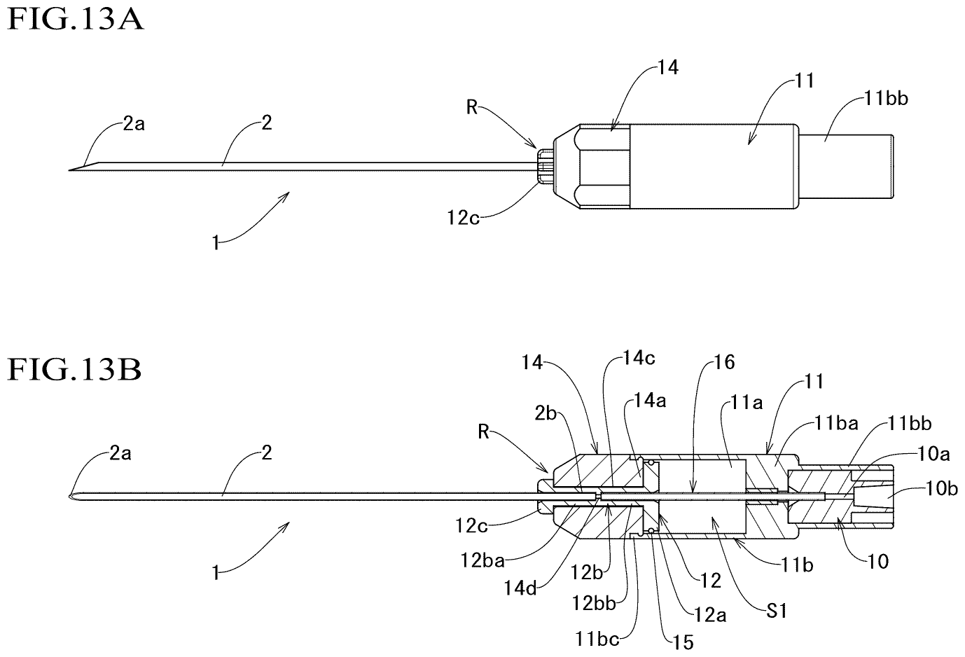

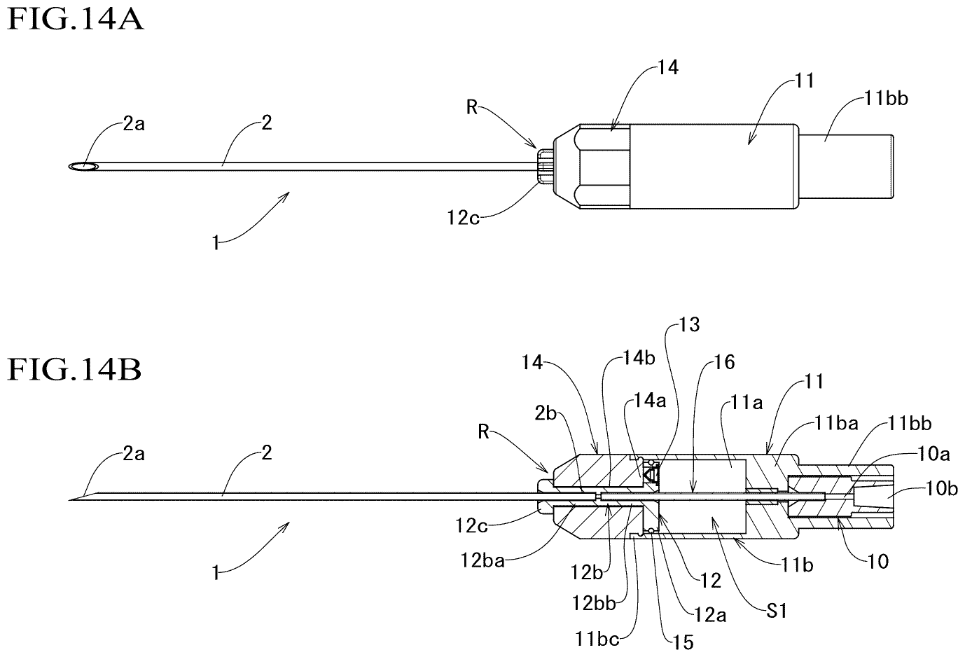

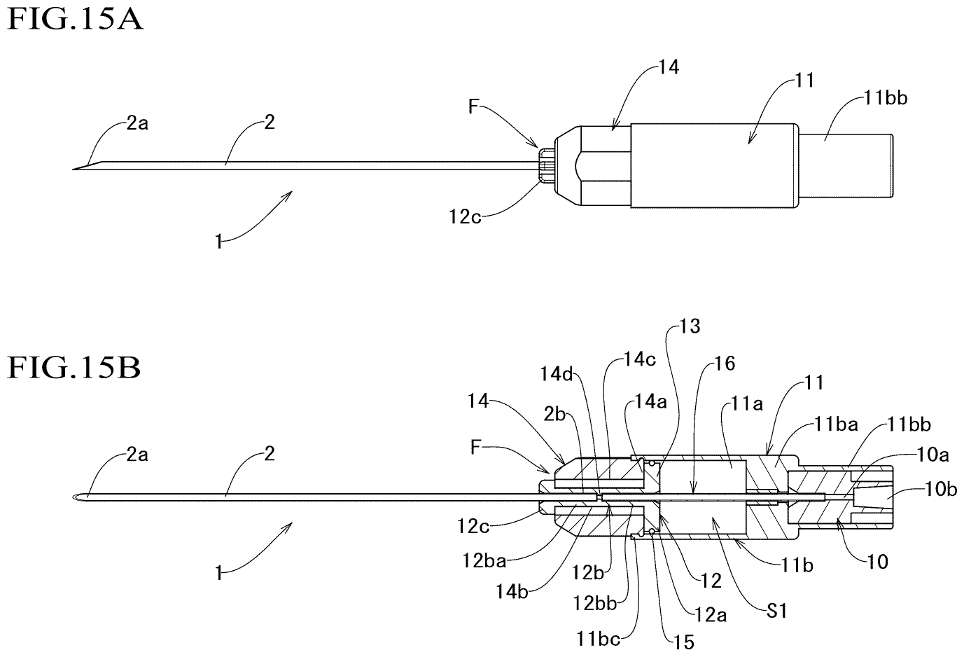

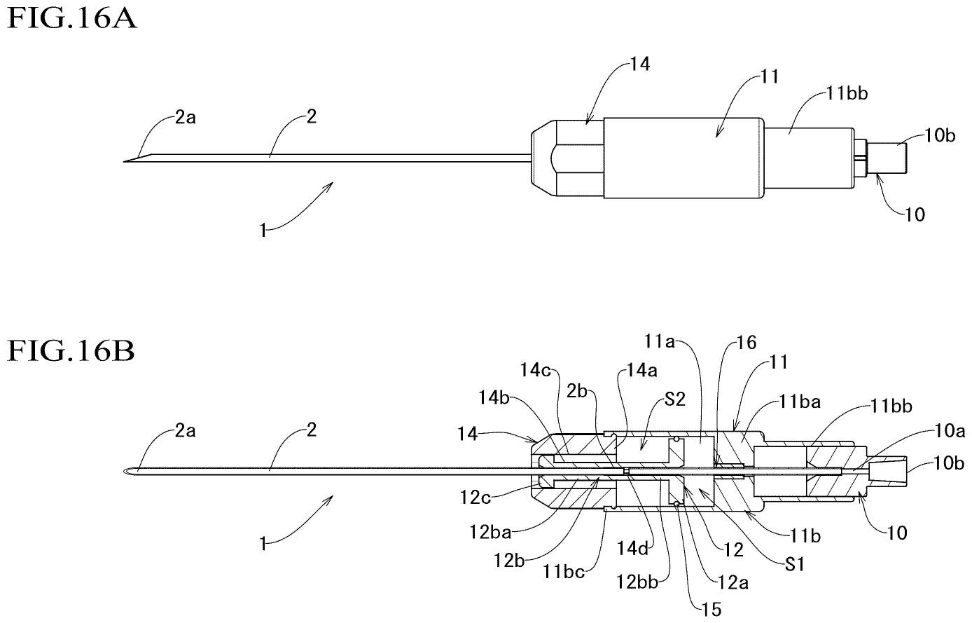

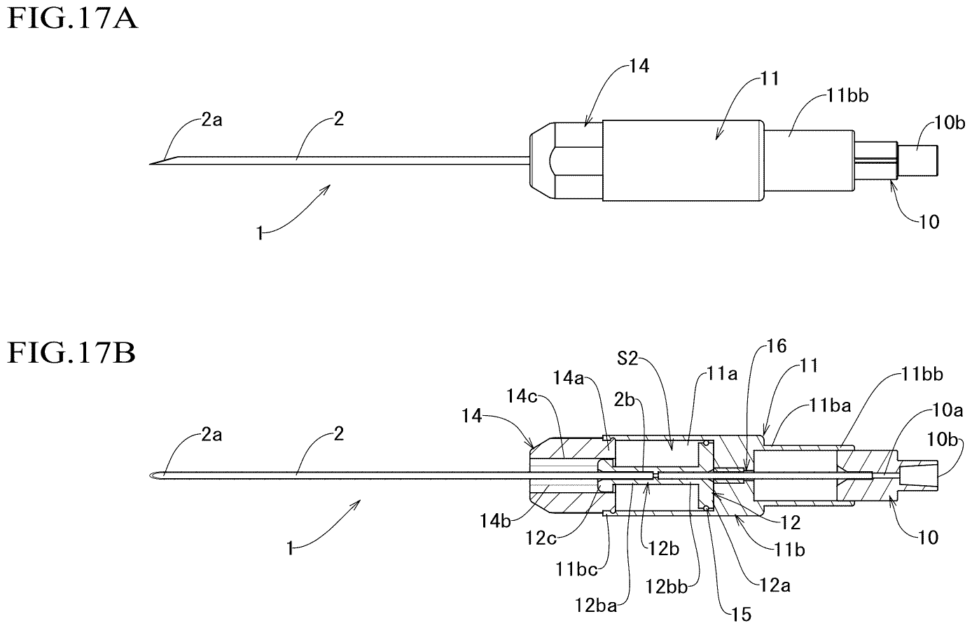

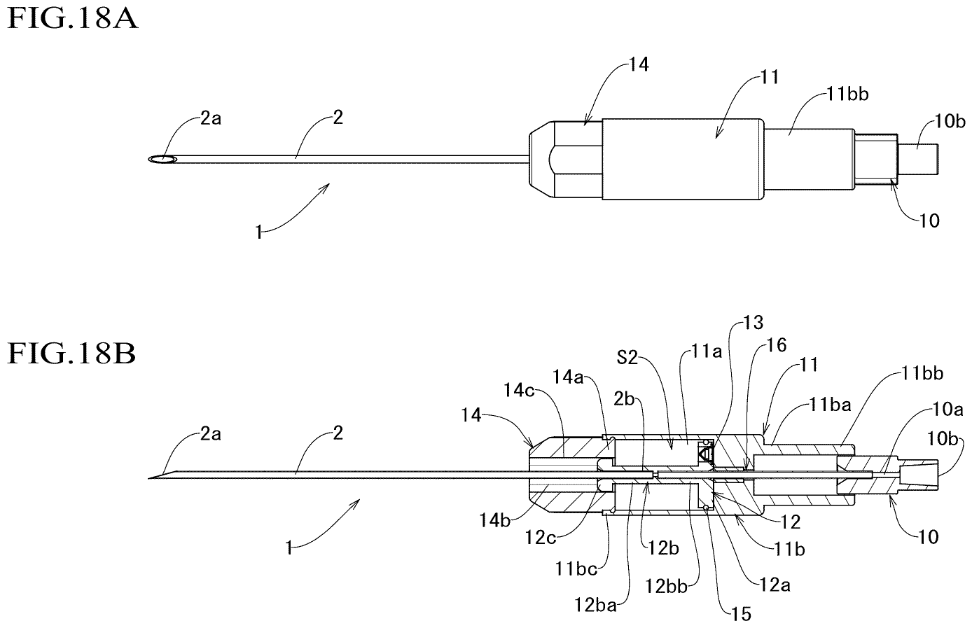

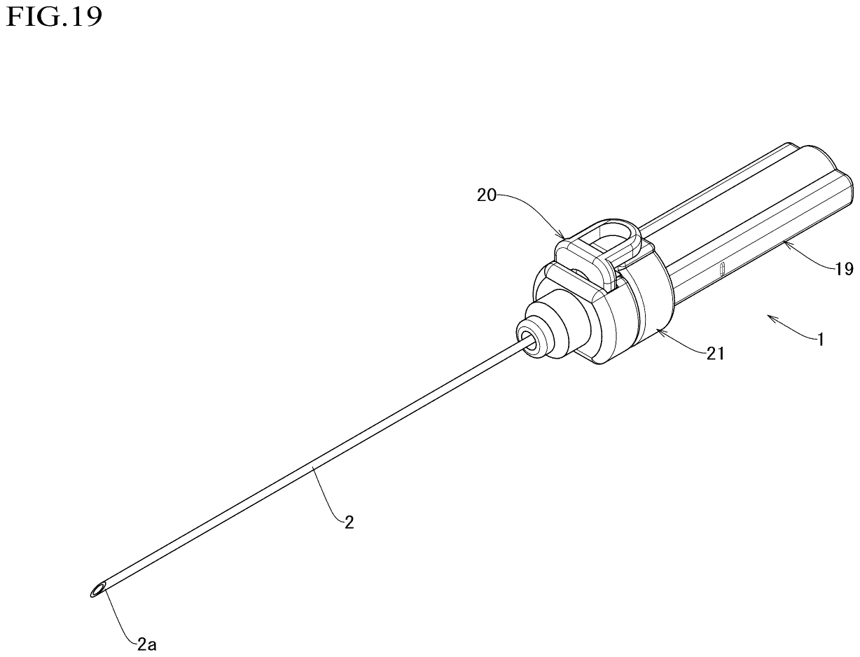

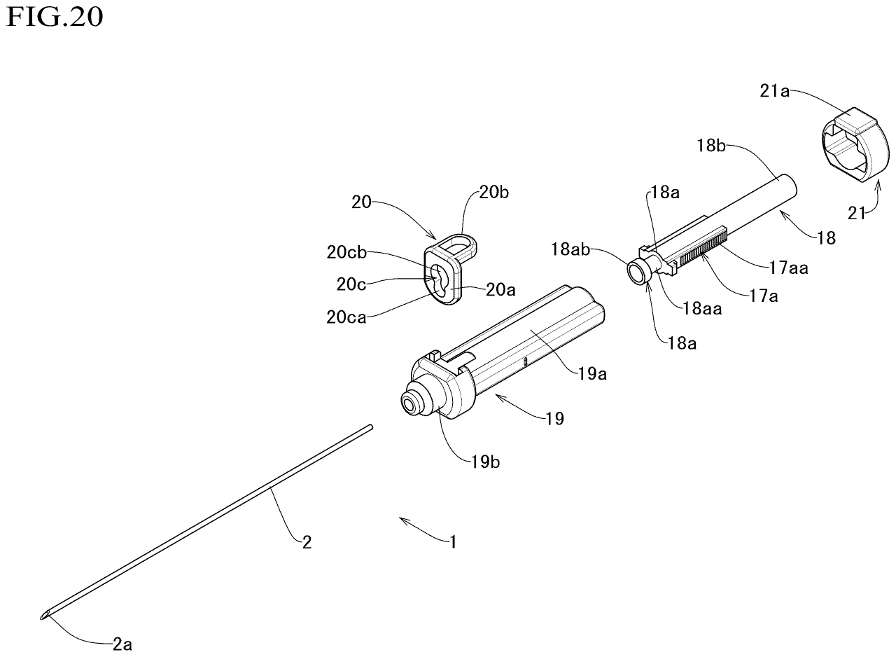

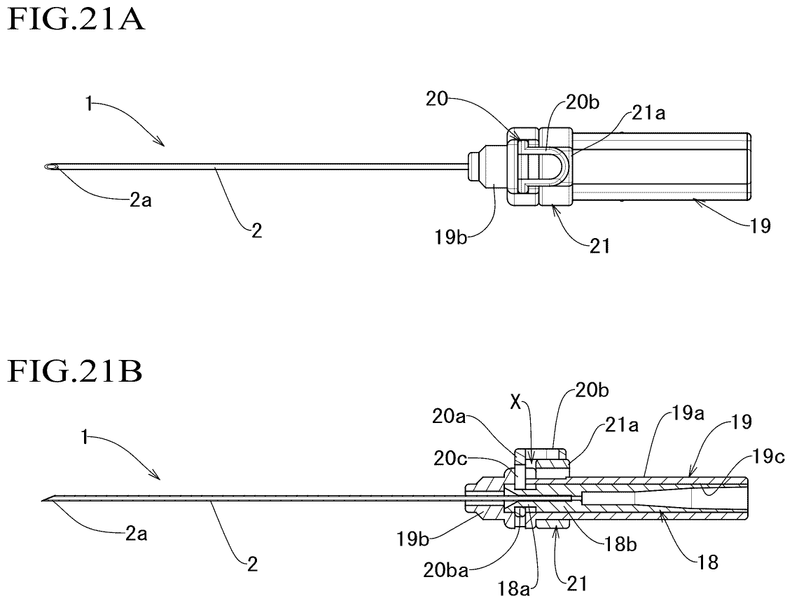

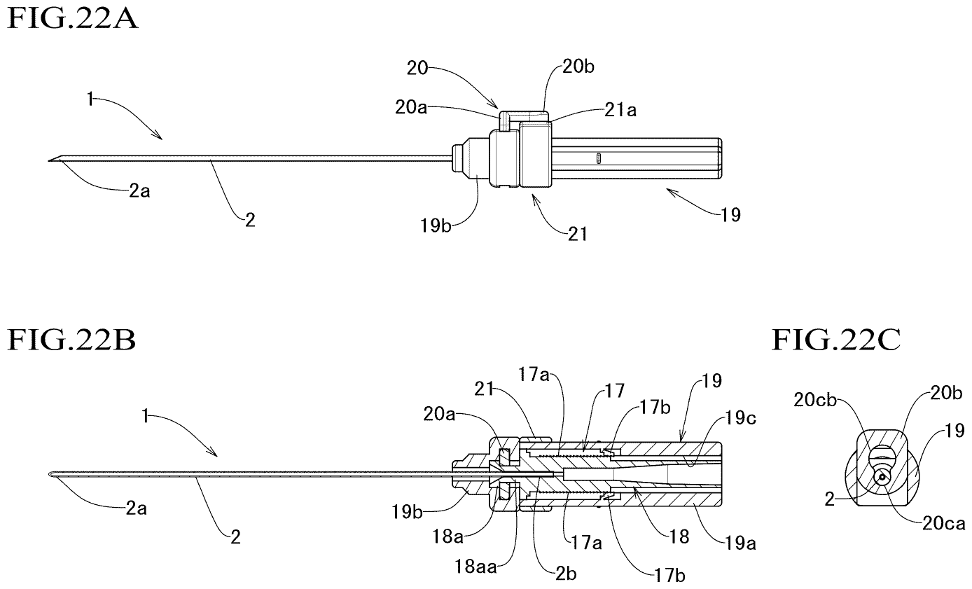

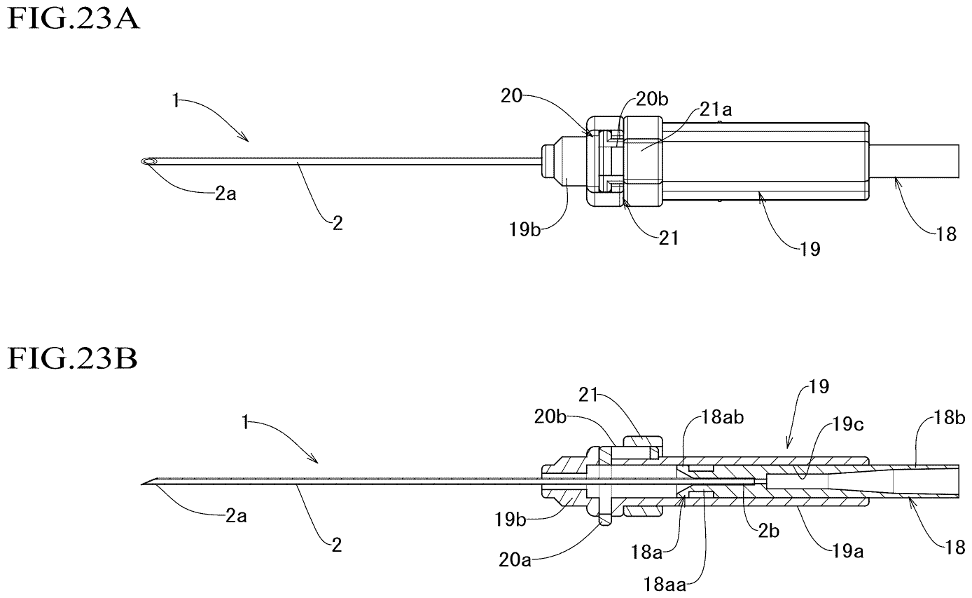

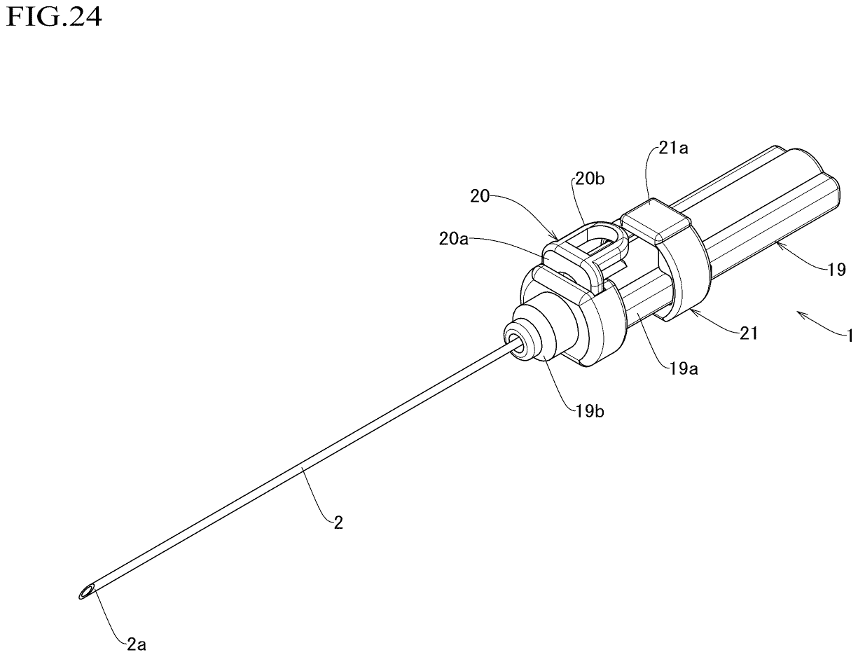

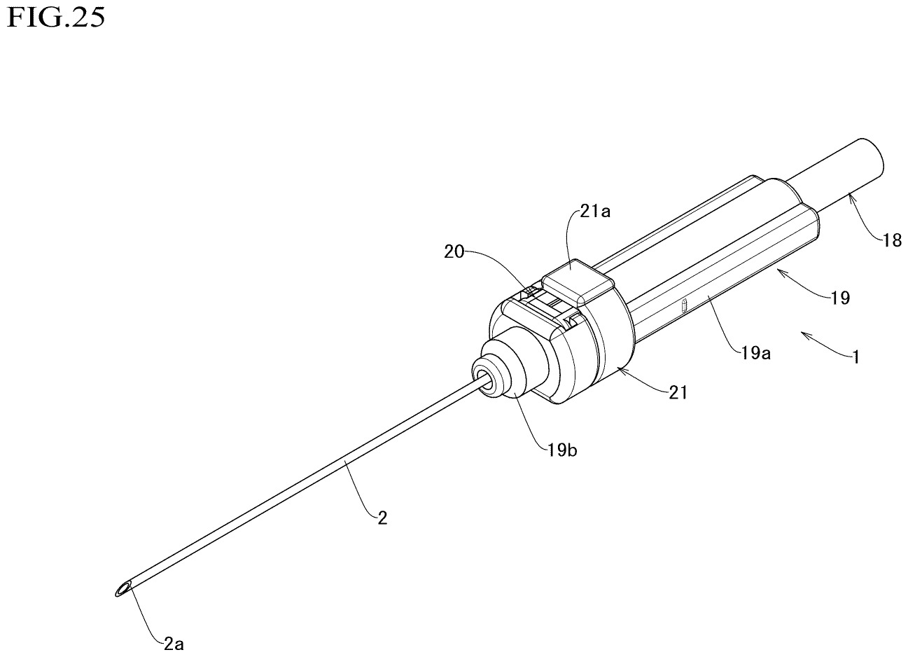

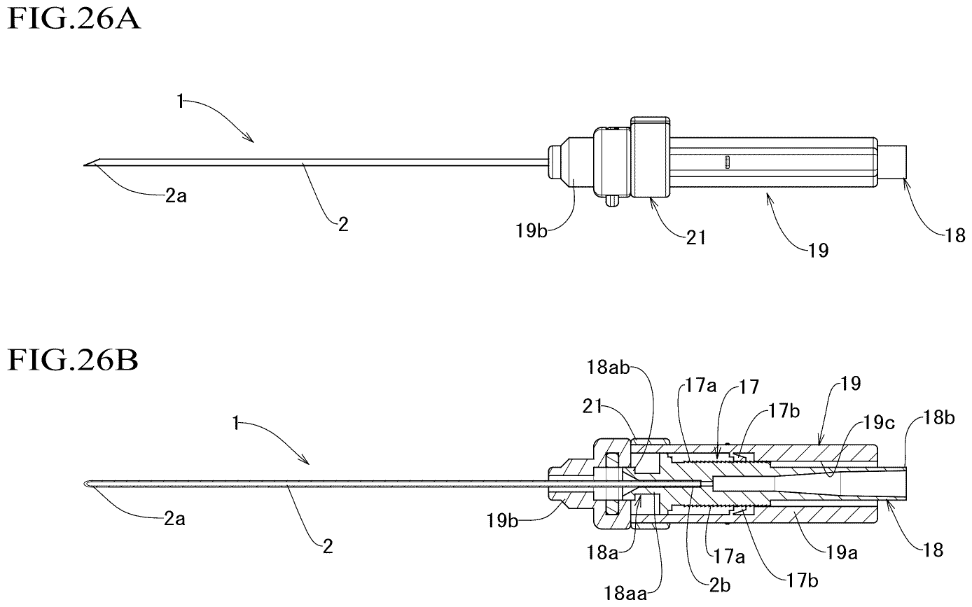

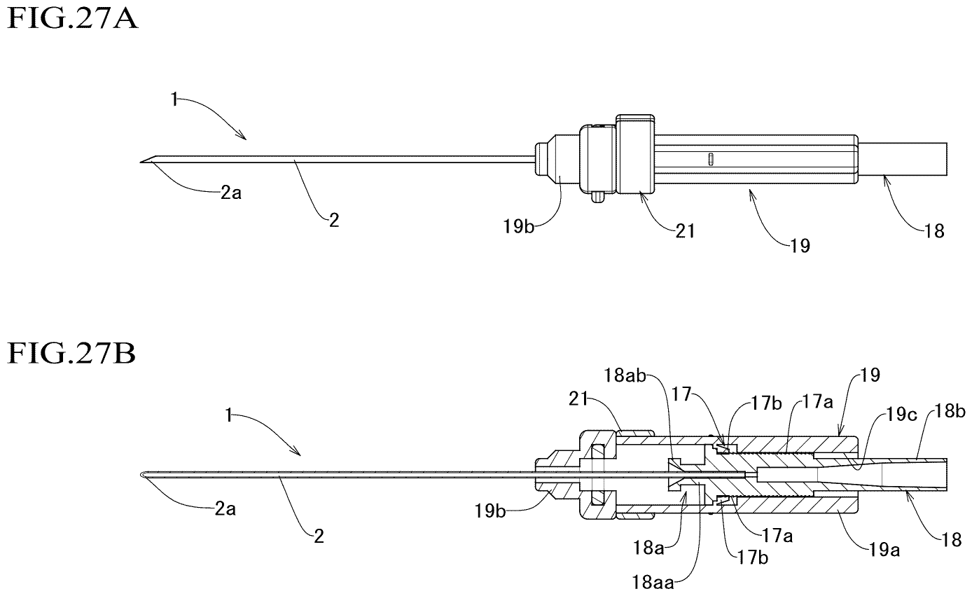

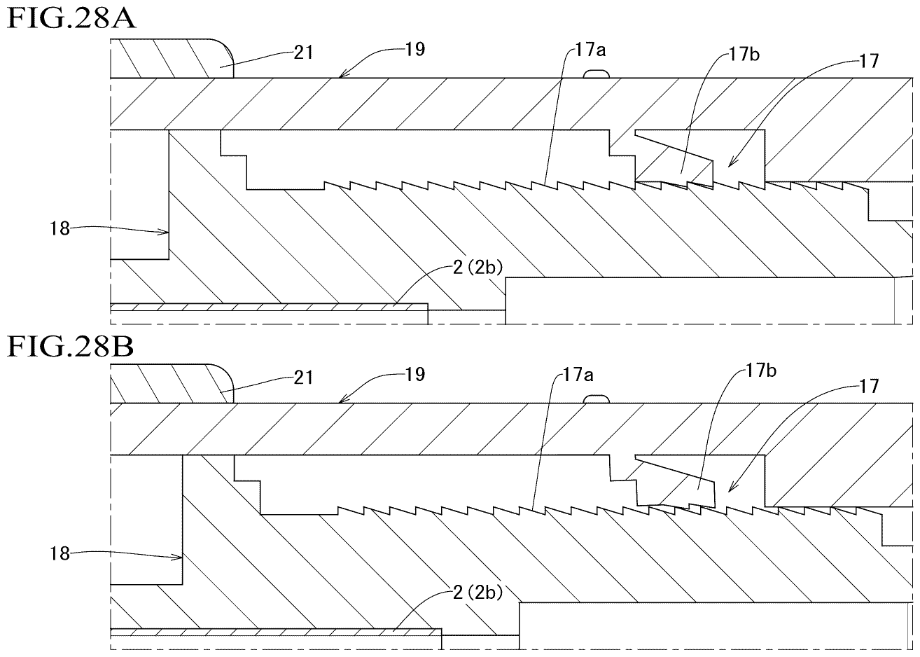

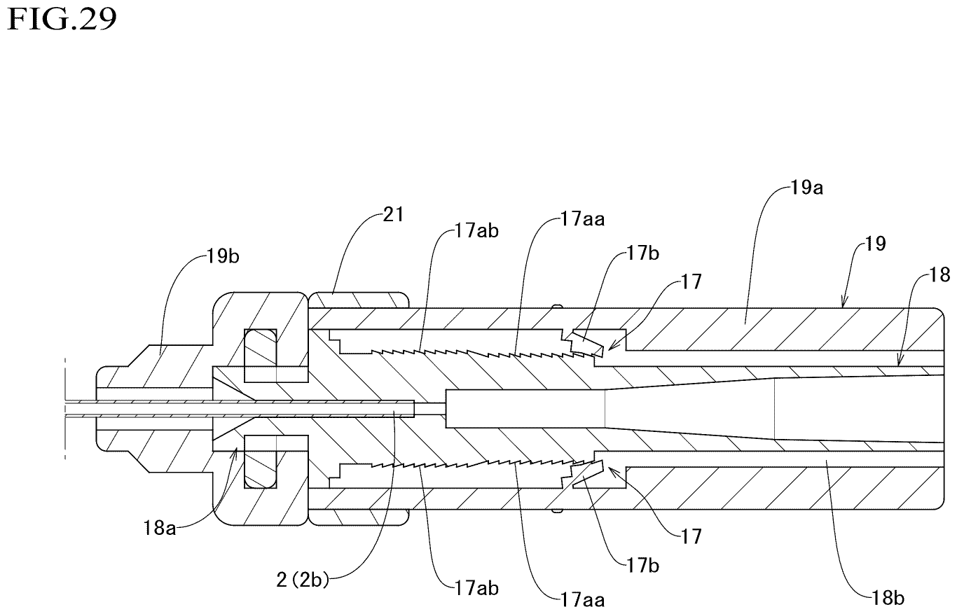

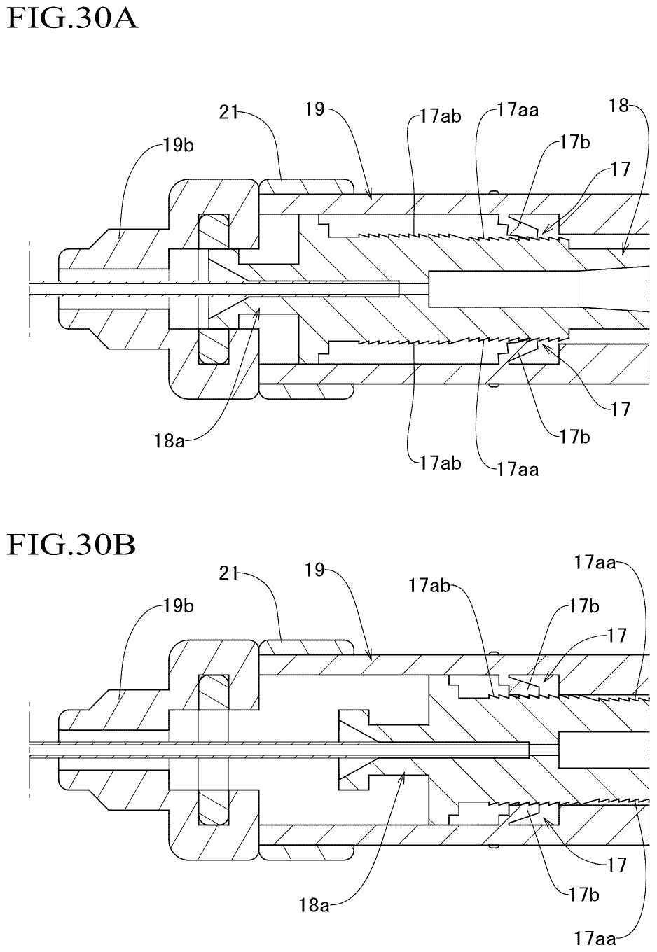

In the fourth embodiment illustrated in FIG. 11 to FIG. 18, as the impact absorption means (puncture pressure reduction means), instead of using the rubber-like elastic material described above, such as a sponge or an urethane material, which contracts when receiving an impact force (pressing force, puncture pressure) to absorb the impact (puncture pressure), a piston mechanism, a ratchet mechanism according to the fifth embodiment as illustrated in FIG. 19 to FIG. 28, or a modification according to the sixth embodiment which uses a ratchet mechanism as illustrated in FIG. 29 and FIG. 30 is also can be used. In the following, these embodiments will be described.

First, the fourth embodiment will be described. The puncturing instrument 1 according to the present embodiment includes the needle main body 2 provided at the tip side and a hub 10 provided at the base end side, in the same manner as those according to the preceding embodiments. As will be described later, the puncturing instrument 1 according to the present embodiment further includes a case body 11 with a cylinder portion 11a formed therein, a movable body 12 with a piston portion 12a that moves within the cylinder portion 11a, and a check valve 13 provided on the piston portion 12a.

The case body 11 includes a case main body 11b having a bottomed cylindrical shape which allows the cylinder portion 11a to be formed therein. On a bottom portion 11ba formed on the base end side of the case main body 11b, a first outer sleeve portion 11bb extending toward the base end side is formed. The hub 10 slidably fits into the first outer sleeve portion 11bb in the axial direction (axial direction of the needle main body 2) and is assembled thereinto, and rotation about the axis is restricted by key-fitting.

On an opening edge portion 11bc of the case body 11b, a base end portion 14a of a tip-side sleeve body 14 extending outward in the axial direction of the needle main body 2 fits and is assembled thereinto in a forcibly fitting manner. This fitting assembly allows rotational movement of the tip-side sleeve body 14 around the axis relative to the case body 11.

The movable body 12 includes the piston portion 12a that moves slidably in the axial direction and around the axis of the needle main body 2 along the inner circumferential surface of the cylinder portion 11a, and on the outer circumferential surface of the piston portion 12a, a rubber O-ring 15 is fit and attached to ensure sealing and sliding performance between the inner circumferential surface of the cylinder portion 11a and the outer circumferential surface of the piston portion 12a. This allows the movable body 12 to move relative to the case body 11 in the axial direction. The relative rotational movement of the movable body 12 around the axis relative to the case body 11 described above is restricted by a communicating tube 16 which is fit and mounted (fixed) to the case body 11.

The movable body 12 includes a main body 12b extending from the piston portion 11a toward the tip side, and a base end portion 2b of the needle main body 2 fits into a tip side half portion 12ba of the main body 12b and is assembled thereinto.

The main body 12b of the movable body 12 fits into a hole sleeve portion 14b formed in the tip-side sleeve body 14 and is assembled thereinto slidably in the axial direction and around the axis, and a locking flange portion 12c having a cross shape in the present embodiment is formed on a tip portion of the movable body main body 12b.

On the hole sleeve portion 14b formed on the tip-side sleeve body 14, a cross-shaped groove hole (key groove) 14c provided for the locking flange portion 12c is formed.

The main body 12b of the movable body 12 fits into the hole sleeve portion 14b of the tip-side sleeve body 14 thus structured in a slidable manner in the axial direction and is assembled thereinto. In the locked state R where the locking flange portion 12c protrudes from the tip of the hole sleeve portion 14b and also is positioned out around the axis from the key groove 14c, the movable body 12 is locked (restricted) so as not to slide in the axial direction.

In this locked state R, by rotating the tip-side sleeve body 14 (case body 11) around the axis relative to the case body 11 so as to cause the groove hole 14c and the locking flange portion 12c to be placed in mutually facing positions, the free state F where the locking flange portion 12c can slide relative to the groove hole 14c is established. This enables the movable body 12 to slide toward the base end side.

A tip portion of the communicating tube 16, whose base end portion fits into the hub 10 and is assembled thereinto while passing through the piston portion 12a, the cylinder portion 11a, and the bottom portion 11ba of the case main body 11b, fits to a base end side half portion 12bb of the movable body main body portion 12b and is assembled thereinto. Thus, the communicating tube 16 functions to integrate the movable body 12 and the hub 10 together with the needle main body 2, and the movable body 12 is allowed to move freely in the axial direction together with the hub 10 whose rotation around the axis is restricted. The needle main body 2 is communicated and connected to a mounting recess hole 10b formed in the hub 10 through a communicating hole 14d provided in the tip-side sleeve body 14, the communicating tube 16, and a communicating hole 10a provided in the hub 10, so as to allow a liquid injection tool such as a syringe (not illustrated) mounted to the mounting recess hole 10b and the needle main body 2 to be communicated with each other.

The piston portion 12a formed on the movable body 12 includes the check valve 13. In the process in which the piston portion 12a moves from the tip side to the base end side of the cylinder portion 11a, within the cylinder portion 11a partitioned by the piston portion 12a, the check valve 13 functions as a one-way opening valve which opens in the direction for causing the air (fluid) to flow from a base end side space S1 toward a tip side space S2 so as to restrict movement of the air in the opposite direction. The valve opening pressure (valve opening force) of the check valve 13 is set in advance, and the check valve 13 functions as the impact absorption means according to the present invention.

For using the puncturing instrument 1, the tip-side sleeve body 14 in the locked state R is rotated relative to the case body 11 in the axial direction to be brought into the free state F where the groove hole 14c matches with the locking flange portion 12c, and then, a needle tip portion 2a of the needle main body 2 is inserted into the patient. The movable body 12 receives the puncture pressure caused by this insertion, and thus receives the load trying to move toward the base end side together with the communicating tube 16 and the hub 10. When this load exceeds the valve opening pressure of the check valve 13, the check valve 13 opens, which allows the air to flow from the base end side space S1 toward the tip side space S2 through the check valve 13. Thus, the cylinder portion 12a causes the piston cylinder portion 11a to relatively move toward the base end side, in other words, the case body 11 moves relative to the needle main body 2 toward the tip side. This prevents the puncture pressure from being excessive and the needle tip portion 2a from entering the body too deeply. Therefore, the puncture pressure can be prevented from being excessive during puncture, and thus puncture with appropriate puncture pressure can be realized.

### Fifth Embodiment

Next, the fifth embodiment illustrated in FIG. 19 to FIG. 28 will be described. This embodiment employs a ratchet mechanism 17 as the impact absorption mechanism. The puncturing instrument 1 according to the fifth embodiment includes the needle main body 2, a hub 18 having a tip portion 18a into which a base end portion 2b of the needle main body 2 fits and is supported, and a case body 19 into which the hub 18 fits and is assembled. In the hub 18, a pair of tooth portions 17a, which functions as the ratchet mechanism 17, is formed on an outer circumferential surface 18b of the tip side half portion so as to protrude radially from the outer circumferential surface 18b, at the positions which are symmetrical relative to the axis of the needle main body 2 on both the sides. Each of the tooth portions 17a includes teeth 17aa, each having a right triangular shape with the tip side being higher than the base end side, which are arranged in a single row along the axis of the needle main body 2.

The hub 18 slidably fits into the case body 19 and is assembled thereinto. The case body 19 includes a main body 19a into which the hub 18 fits and is assembled thereinto, and a tip portion 19b integrally mounted to the tip portion of the main body 19a.

The main body 19a includes a cylindrical portion 19c into which the hub 18 slidably fits and is assembled thereinto. On a middle portion in the axial direction of the cylindrical portion 19c, claw portions 17b, which are locked with the tooth portions 17a of the ratchet mechanism 17, respectively, are formed so as to swing, with protruding from an inner circumferential surface 19ca of the cylindrical portion 19c toward the inside of the cylinder and being biased in the axial direction. Relative movement of the case body 19 relative to the hub 18 toward the tip side is restricted by locking of the claw portions 17b with the tooth portions 17a, in which each of the claw portions 17b comes into contact with the vertical surface (or overhang surface) of each of the teeth 17aa and is locked therewith without passing beyond the top thereof. On the other hand, relative movement thereof toward the base end side is allowed because each of the claw portions 17b moves along the inclined surface of each of the teeth 17aa and thus passes beyond the top thereof, in other words, forcibly passes over it.

The tip portion 19b includes a lock groove 19d, into which a locking body 20 movably fits in the radial direction. The locking body 20 is releasably locked with a neck portion (narrow-diameter portion) 18aa formed on the tip portion 18a of the hub 18.

The locking body 20 is formed in an L-shape and includes a locking flange portion 20a that radially fits into the locking groove 19d in an insertable and removable manner, and an operating flange portion 20b that extends from the radial end portion of the locking flange portion 20a toward the base end side.

The locking flange portion 20a includes a through hole 20c through which the neck portion 18aa passes. The through hole 20c includes a small through hole 20ca and a large through hole 20cb. The small through hole 20ca allows, the neck portion 18aa to pass therethrough but does not allow a head portion (thick-diameter portion) 18ab of the tip to pass therethrough in the locked state R where the operating flange portion 20b is separated from the outer circumferential surface of the case body main portion 19a. The large through hole 20cb allows even the head portion 18ab to pass therethrough in the free state F where the operating flange portion 20b approaches and faces the outer circumferential surface of the case body main portion 19a.

When the locking body 20 is in the locked state R, the hub 18 is restricted by a restricting flange portion 21a so as not to move toward the base end side. On the other hand, when moving toward the base end side and thus is in the free state F, the restricting flange portion 21a moves toward the base end side, which allows the hub 18 to move toward the base end side.

The case body 19 further includes a ring-shaped holding member 21 that fits to the outside of the case body 19 slidably in the axial direction. The holding member 21 includes a restricting flange portion 21a that fits into a gap X between the operating flange portion 20b and the outer circumferential surface of the case body 19 to restrict push-in activation of the operating flange portion 20b for switching the operating flange portion 20b in the locked state to the free state F.

The operating flange portion 20b is designed such that the restricted state, in which the push-in activation of the operating flange portion 20b is restricted by the restricting flange portion 21a, is released when the holding member 21 is moved toward the base end side and the restricting flange portion 21a is pulled out from the gap X. This allows a pushing-in manipulation of the locking body 20 to be performed, and thus the locking body 20 to be operated from the locked state R to the free state F.

When the locking body 20 is pushed in, the operating flange portion 20b of the locking body 20 comes into contact with or approaches and faces the outer circumferential surface of the case body 19. In this state, when the holding member 21, which has moved toward the base end side, is moved toward the tip side, the restricting flange member 21a fits to the outside of the operating flange portion 20b. Thus, the operating flange portion 20b is prevented from moving and protruding toward the locked state R side.

When the locking body 20 is brought into the free state F to make the hub 18 slidable relative to the case body 19 and then the needle main body 2 is inserted into the patient, the needle main body 2 receives the puncture resistance thus generated, which causes relative movement of the hub 18 toward the base end side. In the puncturing instrument 1 designed as described above, however, in the case where the load caused by the puncture resistance exceeds the resistance (reaction force) generated when the claw portions 17b of the ratchet mechanism 17 forcibly pass beyond the tooth portions 17a, the hub 18 moves relative to the case body 19 with the claw portions 17b passing beyond the tooth portions 17a. Thus, puncture with the excessive puncture pressure can be avoided, and therefore, the appropriate puncture can be realized.

Furthermore, in the present embodiment, a physician who is performing the puncture can feel the “click” sensation, which is caused when the claw portions 17b forcibly pass beyond the tooth portions 17a, through his or her fingers holding the puncturing instrument 1. This allows the physician to intuitively recognize that the puncture force has reached an appropriate state.

The ratchet mechanism may be designed to have the structure opposite to that according to the fifth or sixth embodiment in which the tooth portions are formed on the case body side while the claw portions are formed on the hub side.

### Sixth Embodiment

The sixth embodiment employs the ratchet mechanism 17 as the impact absorption mechanism in the same manner as the fifth embodiment. In the ratchet mechanism 17 according to the fifth embodiment, each of the tooth portions 17a has the teeth 17aa of the same height. In this structure, the resistance generated when the claw portions 17b forcibly pass beyond the teeth 17aa is the same at any stage, and thus the same puncture resistance is absorbed. However, in some cases, a physician may desire to perform puncture with a slightly larger puncture resistance after observing the puncture state.

For these cases, according to the sixth embodiment illustrated in FIG. 29 and FIG. 30, as the ratchet mechanism 17, the teeth formed on the tooth portions 17a are designed such that the height of each of tooth portions 17ab at the base end side is lower than that of the teeth 17aa at the tip side. Thus, the resistance generated when the claw portions 17b forcibly pass beyond the teeth increases at the tip side. This enables impact absorption of absorbing a low puncture resistance in the first half of puncture operation while absorbing a high puncture resistance in the latter half, and thus enables selection between a weak puncture resistance state and a strong puncture resistance state based on the puncture condition.

Such adjustment of a puncture resistance can be realized by providing not only two stages, but also, providing three or other multiple stages, as needed, and it may be designed such that the stages may increase consecutively.

### Others

It should be noted that the present invention is not limited to the embodiments described above, and various modifications can be made within the scope of the claims.

### REFERENCE SIGNS LIST

1 puncturing instrument
2 needle main body
3 hub
4 case body
5 impact absorption body (impact absorption means)
7 locking mechanism
10 hub
11 case body
11a cylinder portion
12 movable body
12a piston portion
13 check valve
17 ratchet mechanism
17a tooth portion
17aa teeth
17b claw portion
18 hub
19 case body

1. A puncturing instrument comprising:
a needle main body;
a hub that supports a base end portion of the needle main body; and
a case body that houses the hub, wherein
an impact absorption means for absorbing a puncture pressure during puncture of the needle main body is provided between the case body and the hub.

2. The puncturing instrument according to claim 1, wherein
a locking mechanism that prevents the puncture pressure from being absorbed by the impact absorption means is releasably provided between the case body and the hub.

3. The puncturing instrument according to claim 2, wherein
the hub is mounted rotatably relative to the case body, and
the locking mechanism can be released by rotating the hub relative to the case body.

4. The puncturing instrument according to claim 2, wherein
the locking mechanism is mounted removably relative to the case body, and
the locking mechanism can be released by removing the locking mechanism from the case body.

5. The puncturing instrument according to claim 2, wherein
the locking mechanism is mounted rotatably relative to the case body, and the locking mechanism can be released by rotating the locking mechanism relative to the case body.

6. The puncturing instrument according to claim 1, wherein
the impact absorption means is formed of a member that is deformable during puncture of the needle main body.

7. The puncturing instrument according to claim 6, wherein the impact absorption means is formed of a member that does not restore when deformed.

8. The puncturing instrument according to claim 1, wherein
the impact absorption means is formed with a piston mechanism,
the piston mechanism includes:
a cylinder portion provided in the case body;
a piston portion, to which the needle main body is mounted, provided on a movable body that slidably fits relative to the case body and is assembled thereinto, so as to be movable within the cylinder portion; and
a check valve provided in the piston portion, wherein
the check valve restricts movement of air from a tip side space partitioned by the piston portion provided within the cylinder portion toward a base end side space, while allowing movement of the air from the base end side space toward the tip side space when the puncture pressure reaches a predetermined pressure.

9. The puncturing instrument according to claim 1, wherein
the impact absorption means is formed with a ratchet mechanism,
the ratchet mechanism includes a tooth portion provided on one of a sliding portion of the hub or that of the case body, and a claw potion provided on another one of the sliding portions to be locked with the tooth portion, the sliding portion of the hub and the sliding portion of the case body being designed to move relative to each other, and
locking between the tooth portion and the claw portion are designed such that, for allowing relative movement of the case body, which has received the puncture pressure caused by the puncture of the needle main body, relative to the hub toward a tip side, the claw portion can forcibly pass beyond the tooth portion upon having received the puncture pressure, while, for restricting the relative movement thereof toward a base end side, the claw portion cannot pass beyond the tooth portion.

10. The puncturing instrument according to claim 9, wherein
the tooth portion is formed on a hub side while the claw portion is formed on a case body side.

11. The puncturing instrument according to claim 10, wherein
the tooth portion is designed to have multiple stages with a height increasing from a base end side to a tip side so that the puncture pressure generated when the claw portion forcibly passes beyond the tooth portion becomes higher at the tip side of the tooth portion.
